SEQUENCE LISTING (28 sequences)

SEQ ID NO: 1 - DNA, 2263 bp, Homo sapiens
gtttttctaaggtgaaacagaaagtgtgctcccttcctcttaggctaggtagggcaacaaaaatagcaattgctattgggttacaatttatcaactcattgtgactattctggatggctcttgcttatactggtttaaaaaaacttgagaaatgaaattatgattaggtaactgtagtgtgacagacaagaatctctcttctattaatctatttatgatccacctcaaaccaaagggcagttgagggttgctgtgatttgaatgtgtcccctccaaaattcagatgttgccaatgtgatggtattaagtggtgggcctttaggaggtgattaggccatgaggacttctccttcattaaagagaataaagcccttttaaaagaagctttacatagttccacttttcactttctgccatgtgaggactcagcaagaaggccctaaccagacaccaagtctgctggccccttgatcttggactttccaagactccagaactgtgagaaacaaatgtctgttctttataaattgtcctgtctcaagtatttgttttagcagcacaaaaaggactaagacaagtgttttcttcagtgactgcattatacttaatagatcccactgcattgcaattaccttttggtatacctgcttccctgactactaagggcaaacagcaaatctgactcatgtctaagaccccagtgcctagcatggggcttgcctaagtacatgcttaatgaatcatggaattttatagggaaagaagaccagagagagaaagtggattgggaggaagagattgggacaagaaaataacaagcaagcaaaagaaagagaaaatggtgaaatattgaaatgttcctgggttggttgaactacttcattttatggtaaagcaacagggtccgaaagaggttacctgcctgtagggtgatgctacccaaactttgttgtgcagttctgatgttttcttcttcctctccttaattcttcacctaaggagaaaaagatggaggtagaaagaaaacgtgattattatggcaaacttcctctggggcctggtggagccagcattttgtatgactgtagtttgagatgaactgtagggaaagtgttttttggatcattctttttctagatctgcccagggcccaaaggccaggactctgtccaccctgaccctatctctagatctacactatctattttggtggccgtgagtgacatgtggctgttgagcaccagatgtatggctagatgaaattgagatgtgtagtacatgcaaaatacatgcagatttcaatgccttagtactgaaaaaagaaaaagattgtcaaaaatctaagtaattgtttatattggttgtatgttgaaatgataatagttaagataggttaaagaaaatacattaatagaatcccaccccccatcccccatttctttgtaccttttaaaatgtggtcactagaacatttaaaattctatctgtagcttgccttttttctttcttttttttggacaatgctgctttagatcacccagctgggccctgggcagcacagtccactaggacctagactcgcctcccactcactgcaccttctgcctctattgtcctctagtcctttgcaccatagcacgcctctgtgcagccccctgcctgccacacttcactggctagattctcaggccgtcagtgttgccttttctgtaccttacagctcagcgcaggatggcagaagggttctgagactggggtgacatccagccttgagtccaaactctaacactgattagcttcttaaacttgagaaagacagggcacctatttgaatgctgggttcccttggtttaaaatgaggtaacaataaaaccctacctatttcataggaagttgtcaagatgaaaatcaaaatgaattagtgcatatgaatgtgctttgtaaataatgaaataaggcggggcgcggtggctcacacctgtaatcccagcactttgagaggccgaggcgggtgggtcacgaggttaggagatcgagaacatcctggctaacatggtgaaaccccatctctactaaaaatacaaaaagttagccaggcgtggtggcagccgtctgtagtcccagctactccggaggctgaggcaggagaatggcatgaacccaggaggtggagcttgcagtgagctgagatcgcgccactgcactccagcctgggcaatggagtgagactctgtgtcaaaaaaaaaaaaaaaaaaaaaaaaaa

SEQ ID NO: 2 - DNA, 2924 bp, Homo sapiens
  CDS (10)..(2250)
cttgcagcaatggcttggattagatccacttgcattctcttttttaccttgctttttgcccacatagcagctgtaccgattaagtatcttcctgaagaaaatgtacatgatgcagattttggtgaacagaaggatatttcagaaatcaatttagctgcaggcttggacctctttcaaggggacatcctcttgcagaaatccagaaatggcctgagagacccaaacaccaggtggacgttccccattccttacatcttggctgataatttggggctgaatgctaaaggagccattctgtatgcctttgagatgttccgtctcaagtcctgtgtggatttcaagccctatgaaggagagagctcatatatcatatttcaacagtttgatgggtgctggtctgaggttggtgaccaacatgtgggacagaacatttccattggccaaggatgtgcctataaggccatcatagaacacgagatcctgcatgctttgggattttaccacgagcagtcaaggacggaccgggatgattatgtgaacatctggtgggaccaaattctttcaggttaccagcacaactttgacacctatgatgatagcttaatcacagacctcaatacaccctatgattatgagtctttgatgcactaccagcctttctcatttaacaagaatgcaagtgttcccaccatcacagccaagatccctgagtttaactccattatcggacagcgcctggatttcagtgccattgatttagagaggctgaaccgaatgtacaattgcaccacaactcacactcttttggaccactgtacttttgagaaggcaaacatctgtggaatgattcagggcaccagagatgacactgactgggcccatcaggacagtgctcaggctggagaagtggatcacaccttgttgggacaatgcacaggtgccggctacttcatgcagttcagcaccagctcggggtccgcggaagaggcagccctactggagtctcggattctttacccaaagaggaagcagcagtgcctgcaatttttctataaaatgacgggaagtccttcagacagactcgttgtctgggtcaggagggatgacagcacaggcaatgttcgcaagttggtgaaggtgcagacttttcaaggagatgatgaccacaattggaaaattgcccatgtggtgctcaaagaggaacagaagtttcgctaccttttccagggcacaaaaggcgaccctcagaactcaactgggggaatttacctagatgacatcactctgacagaaaccccctgccccacaggggtctggacagtccggaatttctcccaagtccttgagaacaccagcaaaggggacaagcttcagagccctcgattctacaattcggagggatatggttttggggtaactttatacccaaatagcagagaaagctctggttacttgagacttgcttttcatgtgtgcagtggggagaacgatgctatcctggagtggccggtagaaaacagacaggtgataattaccatccttgaccaggagcctgatgtccggaacaggatgtcctcaagcatggtgttcactacctcgaagtcgcacacatctccagcgataaatgacactgtcatctgggacaggccgtccagggtgggaacctatcatacggactgtaattgttttagaagcatcgacttgggctggagtggtttcatttcccaccaaatgctgaaaaggaggagtttcctgaaaaatgatgacctcatcatatttgtggactttgaagatatcacccacctcagccagactgaagttcccactaaaggcaaaagactgagcccccaaggcctcattctccaaggccaggagcagcaggtctccgaagaaggttcgggaaaggccatgttagaggaagccctacctgtcagcctgagccaggggcagcccagccgacagaagcggtcggtggagaacacaggccccctggaggaccataactggccacagtacttcagagacccatgtgacccaaacccttgccaaaatgacggcatctgtgtgaacgtgaaggggatggcgagctgcaggtgcatctctggacatgctttcttctacacgggggagcgctgtcaggccgtgcaggtgcacggcagtgtcctgggcatggtgatcggaggcacggctggcgtgatcttcttgaccttctccatcatcgccatcctttcccaaaggccaaggaagtgacctgcctgctggcattggccagaccacagcagcacctcctccatgcaggccttaactttcccatggtcaatgcagtttggggcagcttttttatcagccttgctttggataggacctccaaggactaaggcctccagccccatgtgtgacccttgtcatctctctgccccacataattatgttactttgctatgtgctcctaatgtatctagtgtgtcctgtgacaacactcatcacacttcattgtaaatcacttgttttattgactgtctttcctatagactgtaagctccatgagggcaggcacatgttgttctcattgaccgtgctggccccagtgcctagatgcatggctggcacattgttggcactcaacaatggttgaatgaataaaacaataaatgaatgaataactaagatatagaaactctcatttatattgcagattgaatatatatgatgaaattcttatgttgaatatgttagaatcaaatactcatttttcattagatacagtagtgtcatcactcttttaagatcttgttaaagatttcaaataaaggtacttctgggcagccaggctgcacagcatttgctttcctctgagattctaagagaaggcctttaataaatttaataaatattgagttagcaaaaaaaaaaaaaaaaaaaaa

SEQ ID NO: 3 - protein, 746 aa, Homo sapiens
MAWIRSTCILFFTLLFAHIAAVPIKYLPEENVHDADFGEQKDISEINLAAGLDLFQGDILLQKSRNGLRDPNTRWTFPIPYILADNLGLNAKGAILYAFEMFRLKSCVDFKPYEGESSYIIFQQFDGCWSEVGDQHVGQNISIGQGCAYKAIIEHEILHALGFYHEQSRTDRDDYVNIWWDQILSGYQHNFDTYDDSLITDLNTPYDYESLMHYQPFSFNKNASVPTITAKIPEFNSIIGQRLDFSAIDLERLNRMYNCTTTHTLLDHCTFEKANICGMIQGTRDDTDWAHQDSAQAGEVDHTLLGQCTGAGYFMQFSTSSGSAEEAALLESRILYPKRKQQCLQFFYKMTGSPSDRLVVWVRRDDSTGNVRKLVKVQTFQGDDDHNWKIAHVVLKEEQKFRYLFQGTKGDPQNSTGGIYLDDITLTETPCPTGVWTVRNFSQVLENTSKGDKLQSPRFYNSEGYGFGVTLYPNSRESSGYLRLAFHVCSGENDAILEWPVENRQVIITILDQEPDVRNRMSSSMVFTTSKSHTSPAINDTVIWDRPSRVGTYHTDCNCFRSIDLGWSGFISHQMLKRRSFLKNDDLIIFVDFEDITHLSQTEVPTKGKRLSPQGLILQGQEQQVSEEGSGKAMLEEALPVSLSQGQPSRQKRSVENTGPLEDHNWPQYFRDPCDPNPCQNDGICVNVKGMASCRCISGHAFFYTGERCQAVQVHGSVLGMVIGGTAGVIFLTFSIIAILSQRPRK

SEQ ID NO: 4 - DNA, 2434 bp, Homo sapiens
  CDS (113)..(1846)
gttactgtgctctcttccaaggctataggagttctggagctgctggctggagaggagggtggacgaagctctctctagaaagacatcctgagaggacttggcaggcctgaatatgcattggctgcaaaaagttcagggactttgcaccctgtggggtactcagatgtccagccgcactctctacattaatagtaggcaactggtgtccctgcagtggggccaccaggaagtgccggccaagtttaactttgctagtgatgtgttggatcactgggctgacatggagaaggctggcaagcgacccccaagcccagccctgtggtgggtgaatgggaaggggaaggaattaatgtggaatttcagagaactgagtgaaaacagccagcaggcagccaacgtcctctcgggagcctgtggcctgcagcgtggggatcgtgtggcagtggtgctgccccgagtgcctgagtggtggctggtgatcctgggctgcattcgagcaggtctcatctttatgcctggaaccatccagatgaaatccactgacatactgtataggttgcagatgtctaaggccaaggctattgttgctggggatgaagtcatccaagaagtggacacagtggcatctgaatgtccttctctgagaattaagctactggtgtctgagaaaagctgtgatgggtggctgaacttcaagaaactactaaatgaggcatccaccactcatcactgtgtggagactggaagccaggaagcatctgccatctacttcactagtgggaccagtggtcttcccaagatggcagaacattcctactcgagcctgggcctcaaggccaagatggatgctggttggacaggcctgcaagcctctgatataatgtggaccatatcagacacaggttggatactgaacatcttgtgctcacttatggaaccttgggcattaggagcatgcacatttgttcatctcttgccaaagtttgacccactggttattctaaagacactctccagttatccaatcaagagtatgatgggtgcccccattgtttaccggatgttgctacagcaggatctttccagttacaagttcccccatctacagaactgcgtcactgtaggggagtcccttcttccagaaactctggagaactggagggcccagacaggactggacatccgagaatcctatggccagacagaaacgggattgacttgcatggtttccaagacaatgaaaatcaaaccaggatacatgggaacggctgcttcctgttatgatgtacagatcatagatgataagggcaacgtcctgccccccggcacagaaggagacattggcatcagggtcaaacccatcaggcctataggcatcttctctggctatgtggacaatcccgacaagacagcagccaacattcgaggagacttttggctccttggagaccggggaatcaaagatgaagatgggtatttccagtttatgggacgggcagatgatatcattaactccagcgggtaccggattggaccctcggaggtagagaatgcactgatggagcaccctgctgtggttgagacggctgtgatcagcagcccagaccccgtccgaggagaggtggtgaaggcatttgtggtcctggcctcgcagttcctgtcccatgacccagaacagctcaccaaggagctgcagcagcatgtgaagtcagtgacagccccatacaagtacccaagaaagatagagtttgtcttgaacctgcccaagactgtcacagggaaaattcaacgagccaagcttcgagacaaggagtggaagatgtccggaaaagcccgtgcgcagtgagacatctaagagacattcatttggattcccctcttctttctctttcttttccctttgggcccttggccttcctatgatgatatgagattctttatggaagaacatgaatataagttttgtcttgccttggttattagcacaaaactttaccatgttagatgttgaaagaagaaagggaaggaatgagagagagtgaaaaggagagggtaacagaaaaaaaggaaagaaaagtaagtcagggaaatattaaaactgcaagggaaagcaattgaaaaagaaataaagtagggaaagaaggagagaggaagcaagggaaggaggaagaaaggaaagaggagatgaaagggggagaaaagatagaagaaaaataattgaagggagaatcagaaaaataaagagaagaaaggaaagaaataaagagagaaagagaaagaagaaagagcaaaagaacacaagaaagaaagagagggagaaagagagggagaaagggagagaaaaaaattgtaaaaataaaaatagtaaaagaaactgataaaaaaaaaaaaaaaaaaaaaaaaaaaaaaaaaaaaaaaaaaaaaaaaaaaaaaa

SEQ ID NO: 5 - protein, 577 aa, Homo sapiens [sequence omitted]

SEQ ID NO: 6 - DNA, 2378 bp, Homo sapiens
  CDS (39)..(1340)
ctcagtcgtaaagaggaaaggcagaatttttccttgctatggctggaacaaacgcacttttgatgctggaaaacttcatagatggaaaatttttaccttgtagctcatatatagattcttacgacccatcaacaggggaagtgtattgcagagtgccaaatagtggaaaagacgagatcgaagccgcggtcaaggccgccagagaagcctttcccagctggtcatcccgcagcccccaggagcgctcacgggtcctgaaccaggtggcggatttgctggagcagtccctggaggagtttgcccaggccgagtctaaagaccaagggaaaaccttagcactggcaagaaccatggacattccccggtctgtgcagaacttcaggttcttcgcttcctccagcctgcaccacacgtcagagtgcacgcagatggaccacctgggctgcatgcactacacggtgcgggccccggtgggagtcgctggtctgatcagcccctggaatttgccactctacttgctgacctggaagatagctccagcgatggctgcagggaacactgtgatagccaagcccagtgagctgacttcagtgactgcgtggatgttgtgcaaactcctggataaagcaggtgttccaccaggtgtggtcaatattgtgtttggaaccgggcccagggtgggtgaggccctggtgtcccacccagaggtgcccctgatctccttcaccgggagccagcccaccgctgagcggatcacccagctgagcgctccccactgcaaaaagctctccctggagctggggggcaagaatcctgccatcatctttgaggacgccaacctggatgagtgcattccggcaaccgtcaggtccagctttgccaaccaggtcagaagttacgtcaagagagctcttgctgaaggtgcccaaatttggtgcggtgagggagtggataagttgagcctccctgccaggaaccaggcaggctactttatgcttcccacggtgataacagacattaaggatgaatcctgctgcatgacggaagagatatttggtccagtgacgtgtgtcgtcccctttgatagtgaagaggaggtgattgaaagagccaacaacgttaagtatgggctggcggctaccgtgtggtccagcaatgtggggcgcgtccaccgggtggctaagaagctgcagtctggcttggtctggaccaactgctggctcatcagggagctgaaccttcctttcggggggatgaagagttctggaataggtagagagggagccaaggactcttacgacttcttcactgagatcaaaaccatcaccgttaaacactgatctttgctaatggtggagccactatggccaatgcctggctgcaggcatcagttgttcaatgtggtagatgaaaatcatggcatgaattccagctatgccttgacttggcagaaggttatctctagcttatcctcagttcttagtaactttacccactagtgaagagatactgtctattttcaatgtggactcggaaaaaaagacttataagtaggaagatagaacaatgatgccagttgtcaggctcctcccaggttatgttttcatagtgtttctttcatcatcttcattgaactcttgggaatctccagataatcagattatttcatttggtaaattttaaaaaatatgcaatcaggcacagtgcctcatgcctataatcccagcactttgggaggccaaggtgggtggatcacttgagttcaggagttcgagatcagcctaggcaacatggtgaaatcctgtctttaccaaaagtttaaaaattagcttggtgtggtgccctctgcctatagccccagctacttgggaggctgaggtgggaggattgcttgagcccaggcggttgaggctgcagtgagccatgatcattccactgcatttcagcctgggggatacagtgagaccttgtctttaaaaaaaaaatgctgcaaaaccaaaaataaatagccataatttgccatctttggagagattatggagataatctccttgtctattagccactagcatggctaattgatcattgattggacctcattacataagggggctgtggacgtctttggaatttgttaggggaaggctatgtggtaagctctactaaatgtatctgtacaaggatgaagaaaaacaaaataactactacttttggaggaaacttggaacaagaagagtacatagtttgatccattcctacggtcgagtctaatcaattccatttcttagtgtatgcatctaatgaggcttacaacttgctaaccccaaaagaagcaataaatgttaagcaattttagtaaaaaaaaaaaaaaaaaaaaa

SEQ ID NO: 7 - protein, 433 aa, Homo sapiens
MAGTNALLMLENFIDGKFLPCSSYIDSYDPSTGEVYCRVPNSGKDEIEAAVKAAREAFPSWSSRSPQERSRVLNQVADLLEQSLEEFAQAESKDQGKTLALARTMDIPRSVQNFRFFASSSLHHTSECTQMDHLGCMHYTVRAPVGVAGLISPWNLPLYLLTWKIAPAMAAGNTVIAKPSELTSVTAWMLCKLLDKAGVPPGVVNIVFGTGPRVGEALVSHPEVPLISFTGSQPTAERITQLSAPHCKKLSLELGGKNPAIIFEDANLDECIPATVRSSFANQVRSYVKRALAEGAQIWCGEGVDKLSLPARNQAGYFMLPTVITDIKDESCCMTEEIFGPVTCVVPFDSEEEVIERANNVKYGLAATVWSSNVGRVHRVAKKLQSGLVWTNCWLIRELNLPFGGMKSSGIGREGAKDSYDFFTEIKTITVKH

SEQ ID NO: 8 - DNA, 2397 bp, Homo sapiens
  CDS (429)..(1019)
ggcaccaggtctcccaggcttcctcagcgtcgacgccggggagcgccctggggttgggagcgcccggggcccgcgaaggaggaggtggctcaggtgtcagggcgcaccgtgggaaccggccccgggggaggcgtggaaacgcggggcctgggactcgaccagcctctcccgtgcggatcgcaaaatctcggagctgaaacagcccgttgttcgcagcctccctctgacccgccaccctgcacattgttttccattcgcccgggtcgcgggtgggaggagaggcacgccggggttctggagcttggccgcgcgccaggcttgtggccttcgtccccctggggccactggggcggccacgcctctccggcgggaggagagaacgcgtgggtccgggtggctgctccggcccttccgcctccagctcggccatggggtcgcgcagctcccacgccgcggtcattcccgacggggacagtattcggcgagagaccggcttctcccaagccagcctgctccgcctgcaccaccggttccgggcactggacaggaataagaagggctacctgagccgcatggatctccagcagataggggcgctcgccgtgaaccccctgggagaccgaattatagaaagcttcttccccgatgggagccagcgagtggatttcccaggctttgtcagggtcttggctcattttcgccctgtagaagatgaggacacagaaacccaagaccccaagaaacctgaacctctcaacagcagaaggaacaaacttcactatgcatttcagctctatgacctggatcgcgatgggaagatctccaggcatgagatgctgcaggttctccgtctgatggttggggtacaggtgacagaagagcagctggagaacatcgctgaccgcacggtgcaggaggctgatgaagatggggatggggctgtgtccttcgtggagttcaccaagtccttagagaagatggacgttgagcaaaaaatgagcatccggatcctgaagtgactccgtttgtgccttgggcttgctcctgcaaccagtatctccttggaattcatccaaagcccccatggacgcatggacgcagggcgacaataaactgtattttcgtttctaactctatttagggccaagagaagaaagctggaaggatgtgtactaaagtctagctcagcagtccccaacctttttggcatcagggacagtttttccacggatgggtgacaggggatggttttgggatgattcaagtgcattacatttattgtgcactttatttctattatgattacattgtaatatataatgaaataattatacaactcaccataatgtagaatcagcaggagccctgagcttgttttcctgcaattagacggtcccatatgggagtgatgggagacagtgacagatcatcaggcattagattctcataaggagtgcacaatctagatcctttggtgtgcagttcacagtaggatttgggctcctatgataatctaatgccactgctgatctgacaggaggcagagctcaggcggtaatgcaagcaatggggagtggctgtaaatatagatgaagcttcagctcgcctgccgctcaccttgtgctgtgcagcccggttcctaacagaccacagaccccacaccaggtctatctcatttggtctcagagctgtgaatcagccagcaatattttagttgcaaatcactgaaaacccaactcaaagtgacttaagtcagaaagaaattttatgaattcaggtaattaaaaagtccagaagtatctgcctttaggcacagctggatccaagggcacaaatgatgtcatcaggctccagttattctccatctcccagctcagctttttctgtctgtaagcctgattttcaggaaggctctttcctagtgatggagatgaccaccatcagctccaggcttctatcctgctaacccagtaacccagtgggaagagatttacttattccaataattccaagtggagagtgtcattgacccgtttggggtctcatctctacttctaggggaatgaaacactctgagtggccaggcctgtgtcatgtgctaattcctagagccagggaaataaggtctgaggattcaggatggggtgaaaggtggttgcttaaaggaaaatgaaatacaattagcagaataaggggaaacgagtggtctgctctgctcgggcaaaacaagagatgcccattactgtgagggacccttgaagtctggactcttaaatgggtttttgctgattttcctgggtgcatgctaggatgatggggcttgatgcagtagggaagagacgatgtaaaaataataaacaatatataccttcaaaaaaaaaaaaaaaaaaaaaaaaa

SEQ ID NO: 9 - protein, 196 aa, Homo sapiens
MGSRSSHAAVIPDGDSIRRETGFSQASLLRLHHRFRALDRNKKGYLSRMDLQQIGALAVNPLGDRIIESFFPDGSQRVDFPGFVRVLAHFRPVEDEDTETQDPKKPEPLNSRRNKLHYAFQLYDLDRDGKISRHEMLQVLRLMVGVQVTEEQLENIADRTVQEADEDGDGAVSFVEFTKSLEKMDVEQKMSIRILK

SEQ ID NO: 10 - DNA, 2793 bp, Homo sapiens
  CDS (1402)..(1749)
ctttgggcggatcccttgagaccaggagttgaagaccagcctgggcaacatagcaagaccccatctctataggaaattaaacaaaatgtattagacgtgatgacttgtgtttataggctcagctactcgagaggctgaggtgggaggatctcttcagtccaggagtcttcaaggctgcagcaagccatggtggtgccactgcattgcagcccaggtgacagagtgagacattgtctgtaaaaaaagtctttaacttcatggcttattaattctacaaacttggagcaaatttaataatagaataatattacctctattttacatggagaatatacatatagttctacactgaatatagttctacaatggagttctaatgtaatcctggggcagaatgctcgatagataagtaaaaatccaaaatattattgcaaaatttaccctgtctttggtttacttctttactgctgttctacatttttaatttttaggactacttttatacttctcttttgcttataatacctcttggggtagaaaaacatataactgtgacttcaacaatgtttatatatatataattattatgagctatttctacaaccattgttaatatacatctgtcattatctctgagtcactgagttaactagctacataatttatttcccattatagttagctaaaatatattacctagatatattttcagagtatattcagtaatgagactagaaattagaaatgatttgttctggaagctcatctattaaagggcattctggcctttaactaaatcttacacatctttctcttcccattttctgcaatggacatagctcccaaaaagggagaaaatattctttcatgtatttaactacagaatatacctgagtgtgaattctaactttactgtttattagctctatgaaccattcatattttttacactcaattatactatcagcaaatagaagataataatagattaacaatagctaataatgttttgagacaatcaagtgagagtgtatgaaaagcacttatctgagttcctggcacttagacattaaataattacttccctcccttatgtatgaaaggtgactgcccaaatgatgcttggagcgcctctgccctctggcttagcagctgagtgataagataatcgaccatagatttcttaggaatgaccttatccctccttccgagatgattagaaaacattgaattttctgctgatttgtgtgaagccacatggtaagacattgctaattatgatttttacccacgcaggtcagaagttacgtcaagagagctcttgctgaaggtgcccaaatttggtgcggtgagggagtggataagttgagcctccctgccaggaaccaggcaggctactttatgcttcccacggtgataacagacattaaggatgaatcctgctgcatgacggaagagatatttggtccagtgacgtgtgtcgtcccctttgatagtgaagaggaggtgattgaaagagccaacaacgttaagtatgggctggcggctaccgtgtggtccagcaatgtggggcgcgtccaccgggtggctaagaagctgcagtctggcttggtctggaccaactgctggctcatcagggagctgaaccttcctttcggggggatgaagagttctggaataggtagagagggagccaaggactcttacgacttcttcactgagatcaaaaccatcaccgttaaacactgatctttgctaatggtggagccactatggccaatgcctggctgcaggcatcagttgttcaatgtggtagatgaaaatcatggcatgaattccagctatgccttgacttggcagaaggttatctctagcttatcctcagttcttagtaactttacccactagtgaagagatactgtctattttcaatgtggactcggaaaaaaagacttataagtaggaagatagaacaatgatgccagttgtcaggctcctcccaggttatgttttcatagtgtttctttcatcatcttcattgaactcttgggaatctccagataatcagattatttcatttggtaaattttaaaaaatatgcaatcaggcacagtgcctcatgcctataatcccagcactttgggaggccaaggtgggtggatcacttgagttcaggagttcgagatcagcctaggcaacatggtgaaatcctgtctttaccaaaagtttaaaaattagcttggtgtggtgccctctgcctatagccccagctacttgggaggctgaggtgggaggatcgcttgagcccaggcggttgaggctgcagtgagccatgatcattccactgcatttcagcctgggggatacagtgagaccttgtctttaaaaaaaaaaatgctgcaaaaccaaaaataaatagccataatttgccatctttggagagattatggagataatctccttgtctattagccactagcatggctaattgatcattgattggacctcattacataagggggctgtggacgtctttggaatttgttaggggaaggctatgtggtaagctctactaaatgtatctgtacaaggatgaagaaaaacaaaataactactacttttggaggaaacttggaacaagaagagtacatagtttgatccattcctacggtcgagtctaatcaattccatttcttagtgtatgcatctaatgaggcttacaacttgctaaccccaaaagaagcaataaatgttaagcaattttagtaaccagaaaaaaaaaaaaaaaaaaaa

SEQ ID NO: 11 - protein, 115 aa, Homo sapiens [sequence omitted]

SEQ ID NO: 12 - DNA, 1344 bp, Homo sapiens
  CDS (63)..(1187)
ctcaaggacacttgatccactgccagagaggcccagaattttctaacttactgtgtggcagaatgaagcctctgcttgaaaccctttatcttttggggatgctggttcctggagggctgggatatgatagatccttagcccaacacagacaagagattgtggacaagtcagtgagtccatggagcctggagacgtattcctataacatataccaccccatgggagagatctatgagtggatgagagagatcagtgagaagtacaaggaagtggtgacacagcatttcctaggagtgacctatgagacccaccccatgtattatctgaagatcagccaaccatctggtaatcccaagaaaatcatttggatggactgtggaattcacgccagagaatggattgctcctgctttttgccaatggttcgtcaaagaaattctacaaaaccataaagacaactcaagtatacgcaagctccttaggaacctggacttctatgtccttccagttcttaacatagatggttatatctacacttggacaactgatcgtctttggaggaaatcccgttcaccccataataatggcacatgttttgggacggatctcaatcgaaatttcaatgcatcttggtgtagtattggtgcctctagaaactgccaagatcaaacattctgtgggacagggccagtgtctgaaccagagactaaagctgttgccagcttcatagagagcaagaaggatgatattttgtgcttcctgaccatgcactcttatgggcagttaattctcacaccttacggctacaccaaaaataaatcaagtaaccacccagaaatgattcaagttggacagaaggcagcaaatgcattgaaagcaaagtatggaaccaattatagagttggatcgagtgcagatattttatatgcctcatcagggtcttcaagagattgggcccgagacattgggattcccttctcatatacgtttgagctgagggacagtggaacatatgggtttgttctgccagaagctcagatccagcccacctgtgaggagaccatggaggctgtgctgtcagtcctggatgatgtgtatgcgaaacactggcactcggacagtgctggaagggtgacatctgccactatgctgctgggcctgctggtgtcctgcatgtctcttctctaagtgcattctgcccaggcctgctcaaccccagtggcatgagtgtggctggaggaacggtgtgttatggttgtaaagaaaccaaataatttaactaaaaatacttcctatttcaataaggaaaaaaaaaaaaaaaaaaaaaaaaaaaaaaaaaaaaaaa

SEQ ID NO: 13 - protein, 374 aa, Homo sapiens
MKPLLETLYLLGMLVPGGLGYDRSLAQHRQEIVDKSVSPWSLETYSYNIYHPMGEIYEWMREISEKYKEVVTQHFLGVTYETHPMYYLKISQPSGNPKKIIWMDCGIHAREWIAPAFCQWFVKEILQNHKDNSSIRKLLRNLDFYVLPVLNIDGYIYTWTTDRLWRKSRSPHNNGTCFGTDLNRNFNASWCSIGASRNCQDQTFCGTGPVSEPETKAVASFIESKKDDILCFLTMHSYGQLILTPYGYTKNKSSNHPEMIQVGQKAANALKAKYGTNYRVGSSADILYASSGSSRDWARDIGIPFSYTFELRDSGTYGFVLPEAQIQPTCEETMEAVLSVLDDVYAKHWHSDSAGRVTSATMLLGLLVSCMSLL

SEQ ID NO: 14 - DNA, 162 bp, Homo sapiens
gtgaaatctgtctctgtaccagcaggatctttgtccagaagagcatctatagtgaatttttaaagagatttgtagaagctaccagaaagtggaaagtcggcattccctctgatccactggtgagcataggtgctctgataagtaaagcacatttggagaaag

SEQ ID NO: 15 - protein, 566 aa, Rattus norvegicus
MHWLWKIPRLCTFWGTEMFHRSFHMNIKKLMPIQWGHQEVPAKFNFASDVIDHWASLEKAGKRSPGPALWWMNGSGEELKWNFRELSEISKQTANVLTGACGLQRGDRVAVVLPRVPEWWLVTLGCMRSGLVFMPGTTQMKSTDILYRLQSSKARAIVAGDEVVQEVDAVAPDCSFLKIKLLVSEKNREGWLNFKALLKDASPIHQCVETVSQESAAIYFTSGTSGPPKMAEHSHCSLGLKAKMDAGWTGLGPSDTMWTISDTGWILNILGSFLEPWVLGTCIFVHLLPKFDPQTVLKVLSSYPINTLLGAPLIYRMLLQQDLSSYKFPHLHSCFSGGETLLPETLESWKAKTGLEIREIYGQTETGITCRVSRTMKVKPGYLGTAIVPYDVQVIDEQGNVLPPGKEGDMALRVKPIRPIGMFSGYVDNPKKTQANIRGDFWLLGDRGIKDTEGYFHFMGRTDDIINSSGYRIGPSEVENALMEHPAVVETAVISSPDPIRREVVKAFVVLAPEFLSHDQDQLTKVLQEHVKSVTAPYKYPRKVEFVLDLPKTITGKIERAKLRAK

SEQ ID NO: 16 - protein, 196 aa, Mus musculus
MGSRSSHIALIPDVEHIRRETGFSQASLLRLYHRFQALDRDEKGFLSRLDLQQIGALAVNPLGDRIIDSFFPNGSQRLYFAGFARVLAYFRPIDEEDATLRDPKQPEPLNSRMNKLRFAFQLYDLDRDGKISRNEMLQVLRLMVGVQVTDEQLESITDRTVQEADEDGDGAVSFLEFTKSLEKMNIEQKMGIRILK

SEQ ID NO: 17 - protein, 195 aa, Homo sapiens
MGSRASTLLRDEELEEIKKETGFSHSQITRLYSRFTSLDKGENGTLSREDFQRIPELAINPLGDRIINAFFPEGEDQVNFRGFMRTLAHFRPIEDNEKSKDVNGPEPLNSRSNKLHFAFRLYDLDKDEKISRDELLQVLRMMVGVNISDEQLGSIADRTIQEADQDGDSAISFTEFVKVLEKVDVEQKMSIRFLH

SEQ ID NO: 18 - protein, 195 aa, Mus musculus
MGSRASTLLRDEELEEIKKETGFSHSQITRLYSRFTSLDKGENGTLSREDFQRIPELAINPLGDRIINAFFSEGEDQVNFRGFMRTLAHFRPIEDNEKSKDVNGPEPLNSRSNKLHFAFRLYDLDKDDKISRDELLQVLRMMVGVNISDEQLGSIADRTIQEADQDGDSAISFTEFVKVLEKVDVEQKMSIRFLH

SEQ ID NO: 19 - DNA, 1633 bp, Homo sapiens
  CDS (586)..(1461)
gtggatcagcctgctgaactgtcggctagggttataggtggttctgagtccttccggaagctgccgaatcttagaagtcgcgccactgttgtggcgatggaagaaggcgaagctctacagaaaccattttggttgagggaagagtggttttaaagttaatttggaaaggagccggcaagcaaccttcagtcggtgtgcggtttttacagttgaactgtttccagccgctaattatgattgttttagcggttcgtcatctgaaaactgcattctgcacggcggtggtggggggaggacgaggatggagaagcctgggcggacatatttattagctgcggtcctaagtcacgtgtgacattctgtatccagtctatcggttatcccaaggagaacgccctgccggaatggccaatcgccttgagatgtccccgccctattcgccatcttaacatagggcattccccacagctgcaggcaccagtgaagcgtctaagggaccatcttggttgagggcggaagtgaggactgtgtcgcgctggaggagcccacagattctcccggacccacgtggaagcggcactcaagatgctgcgccgcgaggcccgcctgcgccgcgagtacctgtaccgcaaggcccgggaggaggcgcagcgctcagcccaggagaggaaggagcggctgcggcgcgcgctggaagaaaaccgcctgattcccactgagttacgccgagaggctctggccttacaggggtccctggagtttgatgatgctggaggtgaaggtgtgaccagccacgtggatgatgaataccgatgggcaggagtcgaggatcccaaggttatgatcactacctcccgagaccccagttcccgcctcaagatgtttgcaaaggagctgaagctggtgttcccgggcgcccagcgaatgaaccgaggtcgacatgaagtgggggcactggtgcgagcctgcaaagccaacggcgtcaccgatctgctggtcgttcacgagcatcggggcacacctgtggggctcatcgtcagccacctgccctttggtcctactgcctacttcacgctgtgcaatgtggtcatgcggcatgacatcccagacctgggcaccatgtcggaggccaagccccacctcatcacacacggcttctcctcccgcctgggcaagcgggtctctgacatcctccgatacctatttcccgtgcccaaagatgacagccaccgggtcatcaccttcgcaaaccaggacgactacatatcattccggcaccatgtgtataagaagacagaccaccgcaacgtggagctcactgaggtcgggccccgctttgagctgaagctgtacatgatccgtctgggcacgctggagcaggaggccacagcagacgtggagtggcgctggcacccttacaccaataccgcacgcaagagagtcttcctgagcaccgagtgagcacactcaccactcagtcaggacatggacttggaactcaggatggggctgtcatagacagacccaccagtaggaactgtcacagaatggcctgctgaactgggatgtggaactgtggcgggtggagaggtctgaataaaccgtctgtgtcatgaaaaaaaaaaaaaaaaaa

SEQ ID NO: 20 - protein, 291 aa, Homo sapiens
MLRREARLRREYLYRKAREEAQRSAQERKERLRRALEENRLIPTELRREALALQGSLEFDDAGGEGVTSHVDDEYRWAGVEDPKVMITTSRDPSSRLKMFAKELKLVFPGAQRMNRGRHEVGALVRACKANGVTDLLVVHEHRGTPVGLIVSHLPFGPTAYFTLCNVVMRHDIPDLGTMSEAKPHLITHGFSSRLGKRVSDILRYLFPVPKDDSHRVITFANQDDYISFRHHVYKKTDHRNVELTEVGPRFELKLYMIRLGTLEQEATADVEWRWHPYTNTARKRVFLSTE

SEQ ID NO: 21 - DNA, 3800 bp, Homo sapiens
ggaggatctgtgaaatgctaagtgtccaaaataaaatattgctgattgtctttttattgaaagtaaatttcctcattaagccaacctgccttctgtaagtcatagtgcttaaatctcaggatttttcattaggagagacctgtcattaaggatttgtaggtataattgcttagcctccattattggtgcttgggatagagaggttttagatttttctgttttttgttctgcttcaaagctcagtttattgaagacatttataagctattggatcatcacttgaatcaagattttgactagtgagcttaattgtccatttcttatgatttcaaagttacagtctgagaaatggctgattttaataactgtcctttcataaattaatgttggaataaactgaagctggagataaatacttcatgaaaactggctaaagtctgaaataattacctgttttatgtccaatagctactaaatttgatagaacagttttgagaaacatttcattcttgaaggcaacatctgacatggttctcagcaagttggaatagtaaagattgctgggctgttacgatgagtggaaggagcgtgtttgtagcatacaagttattcagcaatcttgtgctgttgacctaaaaatatgtcttaattcttcatcaaggcaagcctggtgaagcctttacttgttactacttcaacagttggaattagttgctctttttacttgatgaaacaaaactatacctctgaatatttatggatactttttactaaatcaggcttgtgttcttaatccaaattagtaaagttttatggaagctgaaatgtaacacagtagtctccccttatctgcaagagatacattccaagactcccagtggatgcctgaaacctcagatagtactgaactccttgttatacagtcatctccccttatctgcaggagatacattccaagaaccctagtggacacctgaaacctcagatagtactgaaccccttgttatacagtcatcttcccttatctgcaggaggagatacattccaagaaccctagtgggtgcctgaaacctcagatagtactgaaccctttatagactatgttttttcaatctgacaaccaaggcgggactaagcggctaaggggcaggtagtacacagtgtggataagcaggacaaagggatgattcacatcccgcacaggacagagcaggagatcatcggatttcatcactcaggacagcttgtgatttattttatttattttttgttttgagatggagtctcactctgtcacccaggctggagtgcagtggtgcagtcttggctcactgcaacctccgcctcctgggttcaagtgattctcctgcctcagcctcccaagtagctgggattacaagtgccctgccccaccacgcccagctaatttttgcatttttagtagagacggggtttcaccaggttggccaggctggtctcgaacttctgacctcgggtgatccacccgcctcatcctcccaaagtgctgggattataggcatgagccaccttgcccggccgacttgtgatttaaaacataaattgtttatttctggaattttccacataatatgtttggaccaaggcttccttggttaacaaactacggaaagtgaaattgcagataaagggggttactgctcctgtgctctaaaactggtgtttgggtgatcaggagcaggtagccaatgggaagagcacatctgagtgataactaaaggagtttctctggtggccttttcacattctccaatgctcaagcatattttccacttaccattttctctttcacctcattttgcctcaccatcccccatccctgcttatttcttaagcccattgatggcactcattaaattgtgtttagggctaatgaatcattgttccttaatatccttttcaatatgccacaatttaggacacatttaaaattttctaaaacaatatcctaatccgaatattgattaatttgagccacattcccaactctaactcagggcacactgccagtcttccccaatatctcctctcaattccccaccacaccttataaaattggaatcaaagaaatctcactctgtcattgtgaatctaagaataaaaacactgattttaatactgctttactagtttcagccttctaagtaggtaggcctctaggtattctgcagatcactggtggtcttgatagccattaatatatgtttgtattatgttattttttaactaaatcacagttggaaaaaaacatctttgatattatgcccttggacctgttactgcatcactggcgcttgtgatgcaataggacacttcacctgtactaaaagggccaagagtaaatgccttgtttttttttttttggtttttgttttgttttgttaaacatgtctatagagttggcagttaatgctgaatttgtcaaacagcccttccaaaactattcttgtatttaaaaaataaatggatctacctaatttctattgattttttttaaaaaagaaatgatcaaattatattcctgagtgactacaccattttatattccaatcaacaatctttagggattccacatcctcagtaacacttggtattgtctgtctttacccactagaattagtgggtgtgaagtgatagctcattgtgatttcagtttgtgtttcttatatcctgtgaccttgcagaactcatttatgagttctaggatttcttctgtagattccttgggattttctctgcagacaatcattatcatctgcaaatagggatggtttttttcttcctttccaatctgcatgttttcatctccttttctttacatactgagatggctagccagaccttgcagaactataggtcatatatccttcctttgtttttattcttaaggggaaggcatccagtctttcaccattaaatatgttattggtagggttttgtttgtttgtttgttttttgtagatactcatgatcagattaagaaagttttcttccattagtgttttctgagcatctttataatgaatgggtgttgaattttgtcaaatgctttttctgcactgactgatatgatcatgtgattttttcttctttagcttgttaatattgtggattgtattcattgatttttttaaacactgaaccaaccactgtccttggaatgaatccagcttggtcatggtataattaatatattgccaaattttatttgctaaaatttgttaaggatttttggatccatattcatgagaaatattgctctgtagttttttcttttaaaattataccatcttggtcagggtttggtgtcaggataacactagcttcataaaatgagttagaattcctcttctaattctagaacagattgtgtagaattggtgttaattcttctttaaatgtttggtagaattttccagtgaaatcgtttggacctggagacttcttttctgatagccttaaaactattgattaaattgcactgtggtctgatagacagtttgttataatttctgttcttttacatttgctgaggagtgctttacttccaactatgtggtcaattttggaataagtgtgatgtggtgctgagaagaatgtacattccgttgatttggggtgaagagttctgtagatgtctattacgtcagcttgatgcagagctgagttcaattcctggatatccttgttaactttctgtctcgttgatctgtcctagagcttaaaagtataatacttgaaaaaaaaaaaaaaaaaaaaaa

SEQ ID NO: 22 - DNA, 2312 bp, Homo sapiens
cagagatctgtggggattctcacaacttccatttctggtgaacagctgaggtcagagaggaggtgggtaccacttgagatgggcctgtggttcccgggtactgtctttccagacacctagaattcccaggttggtagtgaactgagagtcaaccaccatgatggggccagagacagaggaggaagcagtgagtggataagtgcctgtgctgggcagaagggatataggggaaggtggccaaaggtaggatggggaagtggatcccaggaagcttttgtttcttctgaggccaatttaccaccacctcattgggaaattgttggtagggagacacccatccaattgccttgcagaatacagcaaggcagggagcccaagaatcagagccaagggaagaaacgaccagttgaggttagtaatgtgaagtctggcaaccgtgcccgtcattcctccctggttcctctgttcatcaacgttggggctagtctggctggggcatggtgggcctgcctttccatcccttgggccccttggaacaagggggcaactctgctgtgggagatgtagaaaatgggtctcactttctctatttggggcaatagctgctacttttttcctccagatgggtaccatgaggggcagaggagaatctgggatctgtgatgtttagccctctttactgagtcatggctgcctcacgttctagaagtaaggcaggcaaatcattccaggtactgattgtcctggaacctttggctcttccttgacctcatgtcacgtgcgtccatgtgaagagagtccatgaaacgggctttatgtgagcaacaaggctgggtgcaggcaagctgagtccaaaaaaggggtcagcaaagggtggtgggattatcattagttcttatatgtttgggataggcttacaaagtacattctcaagggtggagaaatattacaaagtaccttcttaagggcaggggagaatattacaaagtaccttcttaagggcgggggggagactatattgtatcagggtggggcaggaacaaatcacaatggtggaatgtcatcagttaaggctattttcatttcttttgtggatcttcagttgcttcaggccatctggatgtatacatgcaggtcacagggaatatgatggcttagcttgggttcagaggcctgacacctcaggctgccaaatgtggaagatttaaatacttgaaccaataccctcctcccaaaaactgaaattggcttctgtttctgagttggtccaggcgcaatgttcagcgtatttgaggaaatcacaagaattgtagttaaggagatggatgctggaggggatatgattgccgttagaagccttgttgatgctgatagattccgctgcttccatctggtgggggagaagagaactttctttggatgccggcactacacaacaggcctcaccctgatggacattctggacacagatggggacaagtggttagatgaactggattctgggctccaaggtcagtataaggtagatgacaggtggccagccaggaggccttgaccgtttgtactccatctccttctcaattccccattgttatcaaccaatcaacaacgaaactgtgggatttgtacacatccacacaccttaatttaagaaatgcacggaagagctgcatggtgggaggagagtgggagaagtggagatttatttccactggaaaccgttcctaaatggtcttccttttccattttttcccttgtaaaataatctgcttttaatttagcgagctcttctcatgtgtttatcatttaaatgaataagtaaatgagggcagtttgcttactggttaagaaaggatgcaggctttagggctggaagcacctggtttcaaagcctggctctgcctcttatcagctgcgtaacctttggacaagttgttttattgctctaagtttcagtttcctcctgtgtcaactctagaggactgttgtaagaatcaagtgagggatgggtgcggtggctcactcctgtaatcccagcactttgggaggccgaggtgggcggatcacgaggtcaggagatcgagaccatcctggctaacacagtgaaaccccgtctctacttaaaaaaaaagaatacaaaaaattagccaggcgtggtggcatgcgcctgtagtcctggctactcaggaggctgaggcaagagaattgcttgaacccgggaggtgcaggttgccgtgagctgagatcgcgccactgcactccagcctgggagacaaagcaagcctctgtctcaaaaaaaaaaaaaaaaaaaa

SEQ ID NO: 23 - DNA, 3141 bp, Homo sapiens
  CDS (20)..(2647)
ctatgcagctaccaggacaatggaatctacgttgactttagcaacggaacaacctgttaagaagaacactcttaagaaatataaaatagcttgcattgttcttcttgctttgctggtgatcatgtcacttggattaggcctggggcttggactcaggaaactggaaaagcaaggcagctgcaggaagaagtgctttgatgcatcatttagaggactggagaactgccggtgtgatgtggcatgtaaagaccgaggtgattgctgctgggattttgaagacacctgtgtggaatcaactcgaatatggatgtgcaataaatttcgttgtggagagaccagattagaggccagcctttgctcttgttcagatgactgtttgcagaggaaagattgctgtgctgactataagagtgtttgccaaggagaaacctcatggctggaagaaaactgtgacacagcccagcagtctcagtgcccagaagggtttgacctgccaccagttatcttgttttctatggatggatttagagctgaatatttatacacatgggatactttaaagccaaatatcaataaactgaaaacatgtggaattcattcaaaatacatgagagctatgtatcctaccaaaaccttcccaaatcattacaccattgtcacgggcttgtatccagagtcacatggcatcattgacaataatatgtatgatgtaaatctcaacaagaatttttcactttcttcaaaggaacaaaataatccagcctggtggcatgggcaaccaatgtggctgacagcaatgtatcaaggtttaaaagccgctacctacttttggcccggatcagaagtggctataaatggctcctttccttccatatacatgccttacaacggaagtgtcccatttgaagagaggatttctacactgttaaaatggctggacctgcccaaagctgaaagacccaggttttataccatgtattttgaagaacctgattcctctggacatgcaggtggaccagtcagtgccagagtaattaaagccttacaggtagtagatcatgcttttgggatgttgatggaaggcctgaagcagcggaatttgcacaactgtgtcaatatcatccttctggctgaccatggaatggaccagacttattgtaacaagatggaatacatgactgattattttcccagaataaacttcttctacatgtacgaagggcctgccccccgcatccgagctcataatatacctcatgacttttttagttttaattctgaggaaattgttagaaacctcagttgccgaaaacctgatcagcatttcaagccctatttgactcctgatttgccaaagcgactgcactatgccaagaacgtcagaatcgacaaagttcatctctttgtggatcaacagtggctggctgttaggagtaaatcaaatacaaattgtggaggaggcaaccatggttataacaatgagtttaggagcatggaggctatctttctggcacatggacccagttttaaagagaagactgaagttgaaccatttgaaaatattgaagtctataacctaatgtgtgatcttctacgcattcaaccagcaccaaacaatggaacccatggtagtttaaaccatcttctgaaggtgcctttttatgagccatcccatgcagaggaggtgtcaaagttttctgtttgtggctttgctaatccattgcccacagagtctcttgactgtttctgccctcacctacaaaatagtactcagctggaacaagtgaatcagatgctaaatctcacccaagaagaaataacagcaacagtgaaagtaaatttgccatttgggaggcctagggtactgcagaagaacgtggaccactgtctcctttaccacagggaatatgtcagtggatttggaaaagctatgaggatgcccatgtggagttcatacacagtcccccagttgggagacacatcgcctctgcctcccactgtcccagactgtctgcgggctgatgtcagggttcctccttctgagagccaaaaatgttccttctatttagcagacaagaatatcacccacggcttcctctatcctcctgccagcaatagaacatcagatagccaatatgatgctttaattactagcaatttggtacctatgtatgaagaattcagaaaaatgtgggactacttccacagtgttcttcttataaaacatgccacagaaagaaatggagtaaatgtggttagtggaccaatatttgattataattatgatggccattttgatgctccagatgaaattaccaaacatttagccaacactgatgttcccatcccaacacactactttgtggtgctgaccagttgtaaaaacaagagccacacaccggaaaactgccctgggtggctggatgtcctaccctttatcatccctcaccgacctaccaacgtggagagctgtcctgaaggtaaaccagaagctctttgggttgaagaaagatttacagctcacattgcccgggtccgtgatgtagaacttctcactgggcttgacttctatcaggataaagtgcagcctgtctctgaaattttgcaactaaagacatatttaccaacatttgaaaccactatttaacttaataatgtctacttaatatataatttactgtataaagtaattttggcaaaatataagtgatttttttctggagaattgtaaaataaagttttctatttttccttaagtcccctaaaagccataatttttattattcctttttctcttttttcaattctatgaatatgtattattttaaagttatatttttcacacagagatgatgctatattacaccttcccttttttgttggtttcttaaactctaatctcatgacagattataccttccttattacttgttttatcttactcagaatctttgaatatatttttctgcccagaattatctaaacaaaagggagaacaaaagaagtatgtctcacttgggaactgaatcaactctaaatcagttttgtcacaaaactttttgtatttgactggcaatgctgattaaaattaaaaatgcacagaaaaaaaaaaaaaaaaaaaaaaaaaaaaaaaaaaaaaaa

SEQ ID NO: 24 - protein, 875 aa, Homo sapiens
MESTLTLATEQPVKKNTLKKYKIACIVLLALLVIMSLGLGLGLGLRKLEKQGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLKPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTI

SEQ ID NO: 25 - DNA, 3840 bp, Homo sapiens
gagagctaatcccacccacacccaacacttttggcttttctattccccatacgttttgtaactattctctttcacttatcctttcaagtgaactttctaactggcctgtctgacccagaagatatttaggacattgttattgtgattttattaaatgtataagttaaccttggaagactaaacaacatccttatgatgttaagtttttctatccaaggacatggtttcctttacatttgttcaattttgttttcttcagtagtgttttgtgagttttcctcatataaatcttggacacgtgtgctaaatgtacacttaagtattttattatcattttgctgccatatattggattgtcacttccattttagcttctctctattttttgtctatatacacaaaagctatttttttatatattatttcctactattttactaaattctgttattatttgtagtcatttctccattgatactttggggtttttcagataaataacaattgcatctgaaaataaagattatttttatttccttttcaattccagtgtctcgaattgctttctcttctctaactgcattagctaacacctccatcccaatgttgaaagtaatgagatagtaggcatccttgtcttgttcctgattttggtgacaaagcctttagtgtttcccttaagtaaaatgcaagcttttgagttaaagtatatattttatgtgtagaggaaatccataaatttctttctgttgtgtttttcaacatgaatcagtactaattttgtcaaagtcctattgtgcttttatgaagataatcatgtaattcctcacattagctgtattaatgtgaagaattacattaaaaacggaacaaaatgtgtattcctggaataaaactctctgaattatgatgtattatttttgtgtaatgttcatttggattgcttgccaatattttatttaagatgtatatctcaatattcataaatctaaagttcttggtacaatattaggttttggaaaatacattatacttatttcgtaagaattgatttggaagctttttgtttttatttgtttctagtgctctgaacttatttaagtaatattgggattgactgatctttaaagtttggtagaattccatcagcctggctttgtgcattcagtagcagtgctctgctacttttttcactattctctatttttttaatgtcactcaggtcaattttgataggttgtattttctgagttatccattttatctagattctcatatgtatttgcttaaaattgtgaaaatcatcttgttacttttaaacttttcccccttttcaggttttccctcaaagatgtctaattttgcatatttgtgttttcttccttttgaattgattagattcactagtggcttgtctatttttttattttttattctttcaaaataaaacccaggtttttgtttcttctactatttttctgtattttaatgcattacttttgcttttaccttcctatcaattcctacctttttctttcttttgatttactctgttttcctttttctaactttttaactgggtgtttaattcatttagttttatctcctcatttacataggtacttaatgctataaacttaactgataattcttttagctgtatctcatagattctaacataatatgcattaattatagttactttcaaaatacccaattgattttatttatatttttctttgagtcacgagttgtttagaaacatttttaacttctgagtggaatggtatttggggtttttatttcataaatctctattaaatacctgcattataagaatgctgcctttattattatttctaccagttagcatttattgaggttttcttcatgacctaagatatagagaatatttgtaaaggtttcatatgttcctctaaagttatattctctattattagaattcagagttcagtatatatctacacgatctgtctttatggttattagttctttaatagccttactaactttgttcacttggcatgctataaataagtcacatattagatgtggctatccttaaagtgaatttctctaagtattcttgaatctcttatattttctgctttaagcatgtatctctgtactcggtgcataaatattcattgcttgtgttttaattatccttttgcatatatggtatcctgcttcatctcttaatacttttttggttatgaatgttaccttatcagataccaagatttcctttcccttttttttaatgtggtatttaattgttcaatctctgattttcttaagatttaataaactttttactttaggttagtttcaggtttacagaaaaattgtaagaatattacaggaagttcttctataccctgtacccaatatcctccatttcaacattattgtagcataaccaataaaaccatactgatacttacaatttgaagtccatatattattcagatttccttaatttctacctaatatgcattttctcatccacaatcccatccagggtgccatgttacatcatttcttttcctttttgagacagagtcttgctctgacacccaggctggagtgcagtagtgccatctcggctcactgcaacctctgcctcctaggttcaagtgatcctcctgcctcagcctcccgagtagctgggatgacaaacatgaaccagcacacccggctaatttttatattttttgggagagatgcgtttcaccatgttggccaggctggtctcaaactcctaacctcaagtgatcaacctgcctcagcctcccaaagtgctgggattacaggcgtgagccaccgcacccagccaacattatatttcattgtcacttctctttagactttccttgtttttgatgacattgacagttttaaggaacagtggtctggtattttgtaaactgtccttccatgttagtttgtctgttatttttctcatggttaaacagaattcatgggtttagggaaggaagatcacagagctaagtggtcttccactcatcacatcacatcaaggatgcatgctgtcaatatgacatcactgatgatgttaatcttgatcacctggctgagctgatgttcatcaggtttctccacagtaagattactcttccttctgccccaccctgctctttactgtactctttcaaaagaggtcactaggtcaattccacacttaaggattgggcagttctgttccatctccttgagtggggagaatctgcatacattatctggaattcttctgtacaagagatttgtctattatcccttacatatttgtttattcaatcattcatttacatcagtgtgaattcatagatatttatttcatactttgggacataatccaacagtacattattttgttactcaaattgttctactttggccattgagagctctttccattggttcctgtgttcctttgtttgtttgcttcttttgagcatttcttgctttccgacactacaagatgctttaggctcatcttgtatattccctgccccagccctagaatcagccgtttatccaaggagccctgattcctttcactggagaatggtattggaaacaaagatctagacactggctctgtttgttactattggggtgtctttgcttctaggccctctcagaagacagcgctcagaaatattggccaggcatggtgccccatgcctgtaatcctagcactttgggagggtgagatgggaggatcgtttgaggccaggaattcgggaccagtctggtcaacatagcaaaaccccatctctaagaaaaaaaaaa

SEQ ID NO: 26 - protein, 291 aa, Mus musculus
MLRREARLRREYLYRKAREEAHGSVQEKKERVKRALEENQLIPTELRREALALQGSLEFDDAGGEGVTSHVDDEYRWAGVEDPKVMITTSRDPSSRLKMFAKELKLVFPGAQRMNRGRHEVGALVRACKANGVTDLLVVHEHRGTPVGLIVSHLPFGPTAYFTLCNVVMRHDIPDLGTMSEAKPHLITHGFSSRLGKRVSDILRYLFPVPKDDSHRVITFANQDDYISFRHHVYKKTDHRNVELTEVGPRFELKLYMIRLGTLEQEATADVEWRWHPYTNTARKRVFLSAE

SEQ ID NO: 27 - protein, 269 aa, Mus musculus
RSVQEKKERVKRALEENQLIPTELRRERLALQGSLEFDDAGGEGVTSHVDDEYRWAGVEDPKVMITTSRDPSSRLKMFAKELKLVFPGAQRMNRGRHEVGALVRACKANGVTDLLVVHEHRGTPVGLIVSHLPFGPTAYFTLCNVVMRHDIPDLGTMSEAKPHLITHGFSSRLGKRVSDILRYLFPVPKDDSHRVITFANQDDYISFRHHVYKKTDHRNVELTEVGPRFELKLYMIRLGTLEQEATADVEWRWHPYTNTARKRVFLSAE

SEQ ID NO: 28 - protein, 247 aa, Mus musculus
MLRREARLRREYLYRKAREEAQRSVQEKKERVKRALEENQLIPTELRREALALQGSLEFDDAGGEGVTSHVDDEYRWAGVEDPKVMITTSRDPSSRLKMFAKELKLVFPGAQRMNRGRHEVGALVRACKANGVTDLLVVHEHRGTPVGLIVSHLPFGPTAFTLCNVVMRHDIPDLGTMSEAKPHLITHGFSSRLGKRVSDILRYLFPVPKDDSHRVITFANQDDYISFRHHVYKKTDHRNVELTEVG

(19) United States
(12) Patent Application Publication
(10) Pub. No.: US 2005/0055733 A1
(43) Pub. Date: Mar. 10, 2005

(54) Small intestine and colon genes

(76) Inventors: Zairen Sun, Rockville, MD (US); Xuan Li, Silver Spring, MD (US); Gilbert Jay, North Bethesda, MD (US); Karl F. Kovacs, Rockville, MD (US); Youmin Shu, Potomac, MD (US); Wufang Fan, Germantown, MD (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/482,838

(22) PCT Filed: Jul. 8, 2002
(86) PCT No.: PCT/US02/21242

Related U.S. Application Data
(60) Provisional application No. 60/303,131, filed on Jul. 6, 2001.
(60) Provisional application No. 60/312,486, filed on Aug. 16, 2001.

(51) Int. Cl. C12Q 1/68; A01K 67/00; C07K 14/705

(52) U.S. Cl. 800/8; 435/6; 435/320.1; 435/325; 530/350; 536/23.5

(57) ABSTRACT

The present invention relates to all facets of novel polynucleotides, the polypeptides they encode, antibodies and specific binding partners thereto, and their applications to research, diagnosis, drug discovery, therapy, clinical medicine, forensic science, pathology, and medicine, etc. The polynucleotides are expressed in small intestine or colon and are therefore useful in variety of ways, including, but not limited to, as molecular markers, as drug targets, and for detecting, diagnosing, staging, monitoring, prognosticating, preventing or treating, determining predisposition to, etc., diseases and conditions relating to small intestine or colon.

This application claims the benefit of U.S. Provisional Application No. 60/303,131, filed Jul. 6, 2001, and U.S. Provisional Application No. 60/312,486, filed Aug. 16, 2001, which are hereby incorporated by reference in their entirety.

### DESCRIPTION OF THE DRAWINGS

FIG. 1 shows alignment of SI068 with mouse putative protein BAB25644 (SEQ ID NO 16), human calcium-binding protein P22 (NP_{—}009167; SEQ ID NO 17) and mouse calcium-binding protein P22 (NP_{—}062743; SEQ ID NO 18).

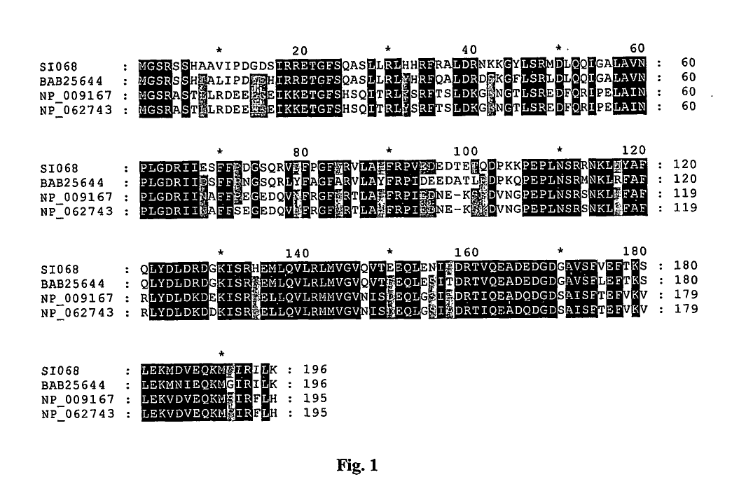

FIG. 2 shows alignment of S1058 and rat kidney-specific protein AF062389 (SEQ ID NO 15).

FIG. 3 shows amino acid sequence homology between Co049 (SEQ ID NO. 20) and AK01334 (SEQ ID NO 26), AK004135 (SEQ ID NO 27), and AK010504 (SEQ ID NO 28).

SEQ ID NOS 1-14 are polynucleotides and polypeptides selective for small intestine, and SEQ ID NOS 19-25 are polynucleotides and polypeptides selective for colon.

### DESCRIPTION OF THE INVENTION

The present invention relates to all facets of novel polynucleotides, the polypeptides they encode, antibodies and specific binding partners thereto, and their applications to research, diagnosis, drug discovery, therapy, clinical medicine, forensic science and medicine, etc. The polynucleotides are expressed in small intestine and colon and are therefore useful in variety of ways, including, but not limited to, as molecular markers, as drug targets, and for detecting, diagnosing, staging, monitoring, prognosticating, preventing or treating, determining predisposition to, etc., diseases and conditions relating to small intestine and colon. The identification of specific genes, and groups of genes, expressed in pathways physiologically relevant to the small intestine and colon permits the definition of functional and disease pathways, and the delineation of targets in these pathways which are useful in diagnostic, therapeutic, and clinical applications. The present invention also relates to methods of using the polynucleotides and related products (proteins, antibodies, etc.) in business and computer-related methods, e.g., advertising, displaying, offering, selling, etc., such products for sale, commercial use, licensing, etc.

Intestine

The small intestine has three major subdivisions, the duodenum, jejunum, and ileum. Like other parts of the gastrointestinal tract, it is comprised of four basic layers or tunics: the mucosa, submucosa, muscularis externa, and serosa. It is the body's major digestive organ, the site where digestion is completed and almost all absorption occurs. The small intestine is highly adapted for nutrient absorption. Both its long length and the modifications of its inner surface provide an extraordinary large surface area enhance absorption enormously. Because of its importance as the energy portal of the body, diseases and conditions that interfere with its function can have great impact on health and maintenance. As a result, identifying intestine markers, gene expression patterns, etc., is of great importance for improving health.

The villi, fingerlike extensions of the inner mucosal surface, are one of the primary specializations characteristic of the intestine's absorption and digestion functions. The epithelial cells that comprise the villi are chiefly absorptive cells or enterocytes. Their capacity to secrete, absorb, and digest specific ions and nutrients, depends on their position along the length of the intestine. The enterocytes, themselves, have microvilli, giving the mucosal surface a fizzy appearance sometimes called the “brush border.” The microvilli comprise enzymes which aid in digestion, such as disacharidases and peptidases. In addition to the enterocytes, mucus-secreting goblet cells and scattered enteroendocrine cells can be identified. Between the villi, the mucosa is studded with pits or openings which lead into tubular intestinal glands called intestinal crypts or crypts of Lieberkuhn The epithelial cells which line the crypts secrete intestinal juice, a fluid mixture comprising mucus. Deep in the crypts are Paneth cells which produce various polypeptides, such as cryptdin, lysozyme, type II (secretory) phospholipase A2, intestinal defensin (e.g., RIP-3). The epithelial cells arise from stem cells at the base of the crypts. They are responsible for the renewal of the villus epithelium, about every three to six days.

The submucosa contains individual and aggregated lymphoid patches, the latter called Peyer's patches. In the duodenum only, mucus-secreting duodenal glands (also called Brunner's glands) are found.

Colon

The colon is a part of the digestive system that functions in the absorption of water, electrolytes, and nutrients that remain after passing through the small intestine, and also in the compaction of feces. The colon is the segment of the gastrointestinal tract which is most affected by tumors, therefore making it an area of intense research. Like the other regions of the intestine, the colon contains four layers or tunic. The serosa is the outermost layer, following by the muscularis, and submucosa. The lining of the colon, and its innermost layer, is the mucosa. The tunica serosa is the outermost covering of the digestive tube. It is comprised of an irregular dense connective tissue surrounded by a mesothelium, a type of squamous epithelium. Underneath the tunica serosa is the muscularis extema, comprising two muscle layers of an inner circular and outer longitudinal muscle. Between the layers are nervous plexus (Auberbach's myenteric). A fibroelastic connective tissue is found at the next level. Called the submucosa, it contains submucosal (Meissner) nervous plexuses, pre- and post-ganglionic parasympathetic fibers, and nonmyelinated preganglionic fibers from the vagus nerve. The innermost layer and lining of the colon is the mucosa. It comprises of an epithelium, a lamina propria, and muscularis mucosae. The epithelium is a simple columnar absorptive epithelium. The lamina propria is a loose connective tissue beneath the epithelium, and the muscularis mucosae is a thin smooth muscle cell layer surrounding the mucosa. The mucosa contains glands or crypts. The crypts comprise goblet cells and regenerative cells or enterocytes. The lamina propria (LP) fills the spaces between the crypts. The crypts are filled with large numbers of goblet cells that secrete mucus to lubricate ejection of the feces.

Nucleic Acids

Si053. Si053 codes for a human meprin, a metalloendopeptidase, containing 746 amino acids. The nucleotide and amino acid sequences of Si053 are shown in SEQ ID NOS 2-3. The polypeptide contains a signal peptide at amino acids (“aa”) and the following domains: a signal peptide at amino acid positions about 1-22; a ZnMc domain at amino acid positions about 71-210; a MAM domain at amino acid positions about 264-433; a MATH domain at amino acid positions about 433-576; and an EGF domain at amino acid positions about 673-710. For more information, see, e.g., Tsukuba and Bond, J. Biol. Chem., 273(52):35260-7, 1998. All or part of Si053 is located in genomic DNA represented by AL161618, NT007402, and BAC Clone RP11-268F1. The present invention relates to any isolated introns and exons that are present in such clone. Such introns and exons can be routinely determined.

Si058. Si058 codes for an AMP-forming synthetase (also known as a ligase) containing 577 amino acids. The nucleotide and amino acid sequences of Si058 are shown in SEQ ID NOS 4-5. An AMP domain is located at about amino acid positions 82-493. Polymorphisms are shown in Table 1.

All or part of Si058 is located in genomic DNA represented by AC027346 and AC003034 and BAC Clones CTD-2519D12 and CIT987SK-A-923A4. The present invention relates to any isolated introns and exons that are present in such clone. Such introns and exons can be routinely determined.

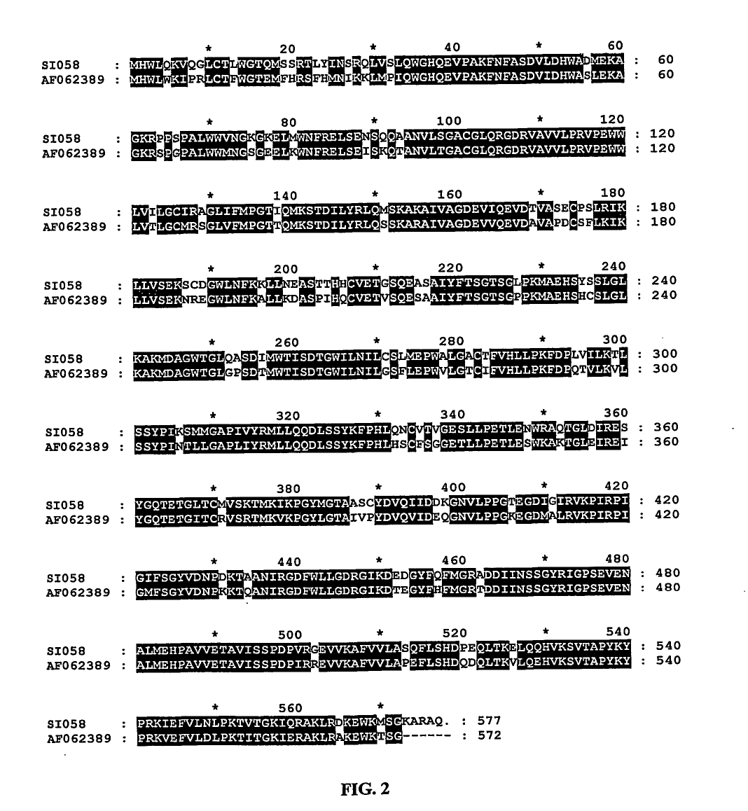

A homolog of Si058 is rat AF062389 (SEQ ID NO 15) which is reported to be expressed selectively in rat kidney. The present invention relates to any conserved domains between the two proteins, polymorphisms in nonconserved regions, etc. FIG. 2 shows the alignment of the two polypeptide sequences. These sequence alignments provide guidance on making mutations in Si058. For instance, residues conserved between the homologs, would be expected to be more important for biological activity, whereas residues which are not conserved, would be expected to be less important for biological activity.

Si058 nucleic acids and the polypeptides they encode are selectively expressed in small intestine. Disorders associated with Si058 can affect small intestine, as well as other tissues and cell types in the body. Such gene effects can be caused by the direct action of the gene on another tissue or cell type, or indirectly, e.g., where a small intestine tissue dysfunction or abnormality has downstream effects on other systems and cell types in the body. Furthermore, low levels of Si058 expression can occur in cell types other than small intestine, and thus can have a function outside of its role in small intestine.

Si058 maps to chromosomal band 16p 12-p13. There are a number of different disorders which have been mapped to, or in close proximity to, this chromosome location. These include, e.g., Inflammatory bowel disease-1 (IBD1). Chromosomal aberrations associated with this locus have been associated with colon cancer and intestinal carcinoid. Other diseases mapped to this locus, include, e.g., medullary cystic kidney disease, retinosa pigmentosa, and microhryocephaly. Nucleic acids of the present invention can be used as linkage markers, diagnostic targets, therapeutic targets, for any of the mentioned disorders, as well as any disorders or genes mapping in proximity to it.

Activity of Si058, and biologically active fragments thereof, can be determined routinely using conventional assay methods. See, e.g., Vessey and Kelley, Biochem. J, 357 (pt. 1):283-288, 2001. Intestinal fatty acid:CoA ligase has been found to differ from kidney, liver, and other ligases. See, e.g., Vessey, Dig. Dis. Sci., 46:438-442, 2001. Si058 is therefore a useful marker for intestine tissue and function.

Si061. Si061 codes for a human aldehyde dehydrogenase (e.g., NM_{—}022568; J. Biol. Chem. 275: 40106-40112, 2000) having 433 amino. The nucleotide and amino acid sequences of Si061 are shown in SEQ ID NOS 6-7. The dehydrogenase domain is located about amino acids 24-433. Si061 has an additional nucleotide sequence not found in other aldehyde dehydrogenases. See, SEQ ID NO 14. Polymorphisms are shown in Table 1. Assays for its activity are well known in the art.

All or part of Si061 is located in genomic DNA represented by NT_{—}023421 and AL021939 and BAC Clone RP3-352A20. The present invention relates to any isolated introns and exons that are present in such clone. Such introns and exons can be routinely determined.

Nucleic acids of the present invention map to chromosomal band 6q22.3-q23.1. There are a number of different disorders which have been mapped to, or in close proximity to, this chromosome location. These include, e.g., Muscular dystrophy congenital merosin-deficient, Syndactyly type III, Oculodentodigital dysplasia, Diabetes mellitus transient neonatal, Rhizomelic chondrodysplasia punctata type 1, Deafness autosomal dominant 10, Metaphyseal chondrodysplasia Schmid type, Hereditary persistence of fetal hemoglobin heterocellular, Atypical mycobacterial infection familial disseminated, BCG infection generalized familial, Tuberculosis susceptibility to, Argininemia, Diabetes mellitus transient neonatal, Muscular dystrophy congenital merosin-deficient, Cardiomyopathy dilated 1F, and Hereditary persistence of fetal hemoglobin heterocellular.

Si068. Si068 codes for a calcium-binding protein containing 196 amino acids. The nucleotide and amino acid sequences of Si068 are shown in SEQ ID NOS 8-9. It has a EF-hand motifs at about amino acid positions 30-58, 115-143, and 156-184. This motif is involved in calcium ion binding. See, e.g., Curr. Opin. Struct. Biol., 10(6):637-43, 2000. It has also been described as a antigen associated with hepatocellular carcinoma (e.g., AF146019 and NM_{—}022097).

All or part of Si068 is located in genomic DNA represented by AC002302 and NT_{—}010604 and BAC clone CT98-SKA-345G4. The present invention relates to any isolated introns and exons that are present in such clone. Such introns and exons can be routinely determined.

The polypeptide coded for by Si068 exhibits sequence identity to mouse (BAB25644; SEQ ID NO 16 and NP_{—}062743; SEQ ID NO 18) and human (NP_{—}009167; SEQ ID NO 17) homologs. These sequence alignments provide guidance on making mutations in Si068. For instance, residues conserved between the homologs, would be expected to be more important for biological activity, whereas residues which are not conserved, would be expected to be less important for biological activity.

Si068 nucleic acids and the polypeptides they encode are selectively expressed in small intestine. Disorders associated with Si068 can affect small intestine, as well as other tissues and cell types in the body. Such gene effects can be caused by the direct action of the gene on another tissue or cell type, or indirectly, e.g., where a small intestine tissue dysfunction or abnormality has downstream effects on other systems and cell types in the body. Furthermore, low levels of Si068 expression can occur in cell types other than small intestine, and thus can have a function outside of its role in small intestine. Nucleic acids of the present invention map to chromosomal band 16p12. There are a number of different disorders which have been mapped to, or in close proximity to, this chromosome location. These include, e.g., inflammatory bowel disease. In addition, an unbalanced chromosomal translation in this region resulted in intestinal adenocarcinoma. Other diseases in this location include, e.g., Liddle syndrome, Pseudohypoaldosteronism type, Convulsions infantile and paroxysmal choreoathetosis, Brody myopathy, Pseudohypoaldosteronism type I, and Arthrocutaneouveal granulomatosis. Nucleic acids of the present invention can be used as linkage markers, diagnostic targets, therapeutic targets, for any of the mentioned disorders, as well as any disorders or genes mapping in proximity to it.

Activity of Si068, and biologically active fragments thereof, can be determined routinely using conventional assay methods. Si068 can be used to measure calcium levels, e.g., by competition assays using radioactive calcium, such as calcium45

Si091. Si091 codes for a zinc-binding carboxypeptidase A metalloprotease containing 374 amino acids. The nucleotide and amino acid sequences of Si091 are shown in SEQ ID NOS 12-13. It has a signal sequence at about amino acid positions 1-21 and a zinc metallopeptidase domain at about amino acids 50-332.

All or part of Si091 is located in genomic DNA represented by AC019052 and BAC Clone RP11-96E16. The gene has nine separate exons. The present invention relates to any isolated introns and exons that are present in such clone. Such introns and exons can be routinely determined.

Si091 nucleic acids and the polypeptides they encode are selectively expressed in small intestine. Disorders associated with Si091 can affect small intestine, as well as other tissues and cell types in the body. Such gene effects can be caused by the direct action of the gene on another tissue or cell type, or indirectly, e.g., where a small intestine tissue dysfunction or abnormality has downstream effects on other systems and cell types in the body. Furthermore, low levels of Si091 expression can occur in cell types other than small intestine, and thus can have a function outside of its role in small intestine.

Si091 maps to chromosomal band 2q34-q35. There are a number of different disorders which have been mapped to, or in close proximity to, this chromosome location. These include, e.g., Ehlers-Danlos syndrome type X, Bjornstad syndrome, Leukemia/lymphoma T-cell, Diabetes mellitus insulin-dependent 13, Myasthenia gravis neonatal transient, Lactic acidosis due to defect in iron-sulfur cluster of complex I, Acyl-CoA dehydrogenase long chain deficiency, Craniofacial-deafness-hand syndrome, Cardiomyopathy, Waardenburg syndrome type III and I, Rhabdomyosarcoma alveolar, Myopathy desminopathic, Acyl-CoA dehydrogenase long chain deficiency, Ichthyosis lamellar type 2, Choreoathetosis familial paroxysmal, Cataract Coppock-like, Amyotrophic lateral sclerosis juvenile, Resistance/susceptibility to TB, and Carbamoylphosphate synthetase I deficiency.

Nucleic acids of the present invention can be used as linkage markers, diagnostic targets, therapeutic targets, for any of the mentioned disorders, as well as any disorders or genes mapping in proximity to it.

Activity of Si091, and biologically active fragments thereof, can be determined routinely using conventional assay methods, e.g., as described in Tan and Eaton, Biochemistry, 1995 May 2;34(17):5811-6; White et al., Biochem Pharmacol, 1986 Aug. 1;35(15):2489-93; Dalle et al., Biochim Biophys Acta, 1999 Oct. 15;1421(2):234-48.

Co049. Co049 is a ribonucleoprotein involved in RNA processing. It contains 291 amino acids. The nucleotide and amino acid sequences of Co049 are shown in SEQ ID NOS 19 and 20. An IMP4-like domain is found at amino acid positions 85-236, and a coiled-coil domain at positions 13-41.

All or part of Co049 is located in genomic DNA represented by AC068137 and NT_{—}005164 and BAC Clone RP11-803A13. The gene has nine separate exons. The present invention relates to any isolated introns and exons that are present in such clone. Such introns and exons can be routinely determined.

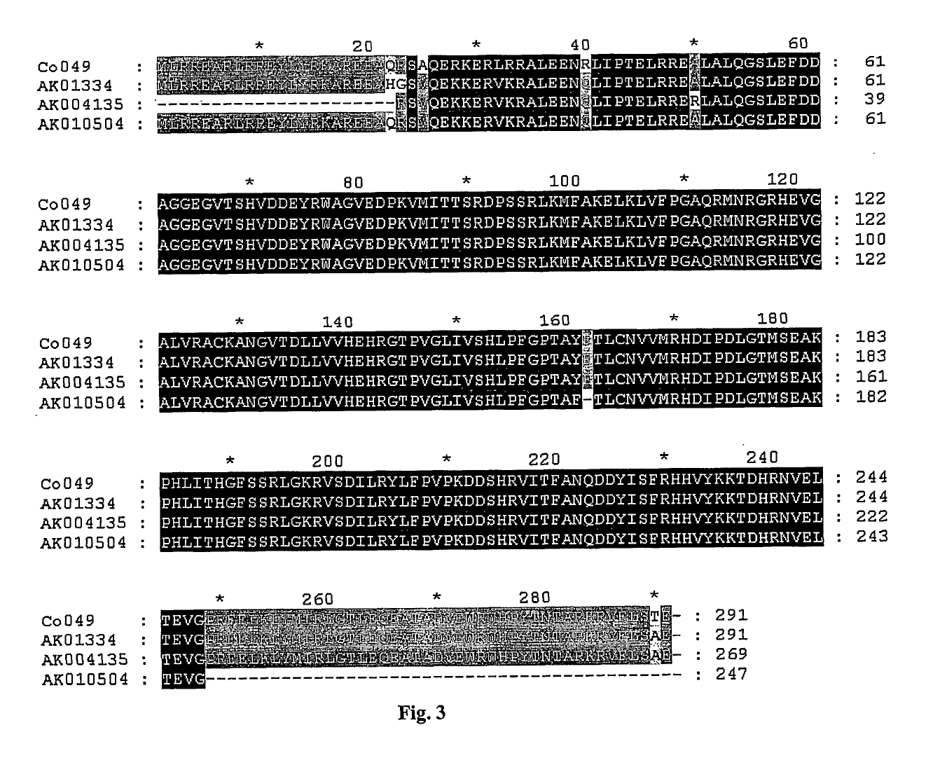

The polypeptide coded for by Co049 exhibits sequence identity to mouse AK01334 (SEQ ID NO 26), AK004135 (SEQ ID NO 27), and AK010504 (SEQ ID NO 28). See, e.g., FIG. 3. These sequence alignments provide guidance on making mutations in Co049 and other ribonucleoproteins. For instance, residues conserved between the homologs, would be expected to be more important for biological activity, whereas residues which are not conserved, would be expected to be less important for biological activity. Amino acid position number 25 of Co049 differs from AK01334, AK004135, and AK010504, and therefore would be expected to be less significant for the biological activity of Co049. RNP activity can be tested routinely, e.g., using in vitro systems for assaying RNA splicing and processing activity. See, e.g., Segualt et al., Mol Cell Biol 1999 Apr., 19(4):2782-90; Dignam et al., Nucleic Acids Res. 11:1475-1489,1983; Lamond, A. I., and B. S. Sproat. 1994, p. 103-140., In D. Rickwood, and B. D. Hames (ed.), RNA processing. A practical approach, Oxford University Press, New York, N.Y.; Ségault, V., C. L. Will, B. S. Sproat, and R. Lührmann, 1995, EMBO J. 14:4010.

Co049 nucleic acids and the polypeptides they encode are selectively expressed in colon. Disorders associated with Co049 can affect colon, as well as other tissues and cell types in the body. Such gene effects can be caused by the direct action of the gene on another tissue or cell type, or indirectly, e.g., where a colon tissue dysfunction or abnormality has downstream effects on other systems and cell types in the body. Furthermore, low levels of Co049 expression can occur in cell types other than colon, and thus can have a function outside of its role in colon.

Nucleic acids of the present invention map to chromosomal band 2q14-q22. There are a number of different disorders which have been mapped to, or in close proximity to, this chromosome location. These include, e.g., Hypothyroidism congenital due to thyroid dysgenesis or hypoplasia, Purpura fulminans, Thrombophilia due to protein C deficiency, Hepatocellular carcinoma, Trichothiodystrophy, Xeroderma pigmentosum group B, Lactase deficiency, Nemaline myopathy-2, and cardiomyopathy. Nucleic acids of the present invention can be used as linkage markers, diagnostic targets, therapeutic targets, for any of the mentioned disorders, as well as any disorders or genes mapping in proximity to it.

Co051. The nucleotide sequence of Co051 is shown in SEQ ID NO. 21 (e.g., with an open reading frame 1792-1968 base pairs. Table 1 lists 6 polymorphisms with respect to known genomic sequences.

All or part of Co051 is located in genomic DNA represented by AL121657 and NT_{—}026235 and BAC Clone CTB-41M14. It is present in an intron of the XDH gene. The present invention relates to any isolated introns and exons that are present in such clone. Such introns and exons can be routinely determined.

Co051 nucleic acids and the polypeptides they encode are selectively expressed in colon. Disorders associated with Co051 can affect colon, as well as other tissues and cell types in the body. Such gene effects can be caused by the direct action of the gene on another tissue or cell type, or indirectly, e.g., where a colon tissue dysfunction or abnormality has downstream effects on other systems and cell types in the body. Furthermore, low levels of Co051 expression can occur in cell types other than colon, and thus can have a function outside of its role in colon.

Nucleic acids of the present invention map to chromosomal band 2p22.3. Colorectal cancer, and hereditary, nonpolyposis is located within this region. There are a number of other disorders which have been mapped to, or in close proximity to, this chromosome location. These include, e.g., Deafness, autosomal recessive; Xanthinuria type I; Muir-Torre syndrome; Glaucoma 3A primary infantile; Ovarian cancer; and Tremor familial essential. Nucleic acids of the present invention can be used as linkage markers, diagnostic targets, therapeutic targets, for any of the mentioned disorders, as well as any disorders or genes mapping in proximity to it.

Co072. The nucleotide sequence for Co072 is shown in SEQ ID NO 22 (e.g., with an open reading frame from 1277-1555 base pairs). Table 1 lists various polymorphisms, including deletions and substitutions, with respect to known genomic sequences. The polypeptide coded for by Co072 exhibits sequence identity to colony stimulating factor, KIAA0130, and Pro2521.

All or part of Co072 is located in genomic DNA represented by AC019095 and NT_{—}010685, and BAC Clone RP1-387H17. The present invention relates to any isolated introns and exons that are present in such clone. Such introns and exons can be routinely determined.

Co072 nucleic acids and the polypeptides they encode are selectively expressed in colon. Disorders associated with Co072 can affect colon, as well as other tissues and cell types in the body. Such gene effects can be caused by the direct action of the gene on another tissue or cell type, or indirectly, e.g., where a colon tissue dysfunction or abnormality has downstream effects on other systems and cell types in the body. Furthermore, low levels of Co072 expression can occur in cell types other than colon, and thus can have a function outside of its role in colon.

Nucleic acids of the present invention map to chromosomal band 17q21.1. There are a number of different disorders which have been mapped to, or in close proximity to, this chromosome location. These include, e.g., Elliptocytosis Malaysian-Melanesian type, Gliosis familial progressive subcortical, Hemolytic anemia due to band 3 defect, Hypertension essential 145500, Pseudohypoaldosteronism type II, Renal tubular acidosis distal, Spherocytosis hereditary, Symphalangism proximal, Acanthocytosis one form, Epidermolytic hyperkeratosis, Meckel syndrome, Mulibrey nanism, Acetyl-CoA carboxylase deficiency, White sponge nevus, Pachyonychia congenita Jadassohn-Lewandowsky type, Epidermolysis bullosa simplex Koebner Dowling-Meara and Weber-Cockayne types, Epidermolysis bullosa simplex recessive, and Epidermolytic palmoplantar keratoderma, Sanfilippo syndrome type B, Pachyonychia congenita Jackson-Lawler type, Wilms tumor type 4, breast cancer, Glycogen storage disease I, Ovarian cancer, Parkinsonism-dementia with pallidopontonigral degeneration, Nonepidermolytic palmoplantar keratoderma, and Dementia frontotemporal with parkinsonism. Nucleic acids of the present invention can be used as linkage markers, diagnostic targets, therapeutic targets, for any of the mentioned disorders, as well as any disorders or genes mapping in proximity to it.

Co127. Co127 codes for an ectonucleotide pyrophosphate/phosphodiesterase 3 (ENPP3; NM_{—}005021) containing 875 amino acids. See, e.g., Bollen et al., Crit. Rev. Biochem. Mol. Bio., 35(6):393-432, 2000. The nucleotide and amino acid sequences of Co 127 are shown in SEQ ID NOS 23 and 24. It contains a signal peptide at about amino acid positions 1-39, somatomedin B-like (“SO”) domains at amino acid positions 50-93 and 94-134, a phosphodiesterase domain at amino acid positions 1410-510, and an endonuclease domain (“NUC”) at amino acid positions 627-857. A polymorphism is listed in Table 1.

All or part of Co127 is located in genomic DNA represented by AC005587 and NT_{—}023579 and RP-998G15. The gene contains at least 19 exons. The present invention relates to any isolated introns and exons that are present in such clone. Such introns and exons can be routinely determined.

Co127 nucleic acids and the polypeptides they encode are selectively expressed in colon. Disorders associated with Co127 can affect colon, as well as other tissues and cell types in the body. Such gene effects can be caused by the direct action of the gene on another tissue or cell type, or indirectly, e.g., where a colon tissue dysfunction or abnormality has downstream effects on other systems and cell types in the body. Furthermore, low levels of Co127 expression can occur in cell types other than colon, and thus can have a function outside of its role in colon.

Nucleic acids of the present invention map to chromosomal band 6q22.31-q23.3. There are a number of different disorders which have been mapped to, or in close proximity to, this chromosome location. These include, e.g., Metaphyseal chondrodysplasia Schmid type, Hereditary persistence of fetal hemoglobin heterocellular, Atypical mycobacterial infection familial disseminated, BCG infection generalized familial, Tuberculosis susceptibility to, Argininemia, Diabetes melltus transient neonatal, Muscular dystrophy congenital merosin-deficient, Cardiomyopathy dilated 1F, Hereditary persistence of fetal hemoglobin heterocellular, Heterotaxia visceroatrial autosomal recessive, Deafness autosomal dominant, and genetic loci control human predisposition to schistosomiasis. Nucleic acids of the present invention can be used as linkage markers, diagnostic targets, therapeutic targets, for any of the mentioned disorders, as well as any disorders or genes mapping in proximity to it.

Co144. The nucleotide sequence of Co144 is shown in SEQ ID NO 25 (e.g., with an ORF from 193-441 base pairs). Polymorphisms are listed in Table 1. All or part of Co144 is located in genomic DNA represented by AC020549 and NT_{—}009338 and BAC Clone RP11-172C16. The present invention relates to any isolated introns and exons that are present in such clone. Such introns and exons can be routinely determined.

Co144 nucleic acids and the polypeptides they encode are selectively expressed in colon. Disorders associated with Co144 can affect colon, as well as other tissues and cell types in the body. Such gene effects can be caused by the direct action of the gene on another tissue or cell type, or indirectly, e.g., where a colon tissue dysfunction or abnormality has downstream effects on other systems and cell types in the body. Furthermore, low levels of Co144 expression can occur in cell types other than colon, and thus can have a function outside of its role in colon.

Nucleic acids of the present invention map to chromosomal band 11q23. There are a number of different disorders which have been mapped to, or in close proximity to, this chromosome location. These include, e.g., Hypoalphalipoproteinemia, Vitreoretinopathy exudative familial, Paraganglioma familial nonchromaffin, Thrombocytopenia Paris-Trousseau type, Leukemia myeloid/lymphoid or mixed-lineage, Jacobsen syndrome, Immunodeficiency T-cell receptor/CD3 complex, Immunodeficiency due to defect in CD3-gamma, Hypertriglyceridemia, Corneal clouding autosomal recessive, Charcot-Marie-Tooth neuropathy-4B, Breast cancer-3, Amyloidosis 3 or more types, ApoA-I and apoC-III deficiency combined, and erythrocytosis. Nucleic acids of the present invention can be used as linkage markers, diagnostic targets, therapeutic targets, for any of the mentioned disorders, as well as any disorders or genes mapping in proximity to it.

The present invention relates to polynucleotides, such as DNAs, RNAs, and fragments thereof, which are expressed in small intestine or colon. These sequences are preferably selectively expressed in small intestine or colon, as compared to other tissues. SEQ ID NOS 1-14 and 19-25 show nucleotide sequences of polynucleotides in accordance with the present invention. By the phrase “selectively expressed,” it is meant that a nucleic acid molecule comprising the defined sequence of nucleotides, when produced as a transcript, is characteristic of the tissue or cell-type in which it is made. This can mean that the transcript is expressed only in that tissue and in no other tissue-type, or it can mean that the transcript is expressed preferentially, differentially, and more abundantly (e.g., at least 5-fold, 10-fold, etc., or more) in the small intestine or colon when compared to other tissue-types. In either case, a selectively expressed polynucleotide is a useful small intestine or colon marker and probe because its occurrence in a sample indicates the presence of small intestine or colon, having significant applications in diagnosis, therapy, histology, pathology, forensics, transplantation, and related areas.

SEQ ID NOS 1-14 and 19-25 show various nucleotide sequences for each selective polynucleotide of the present invention and corresponding polypeptide sequences. A selectively expressed polynucleotide is useful in a variety of different applications as described in greater details below. Because it is more abundant in small intestine or colon, it (or the polypeptide encoded by it) can be used as a diagnostic to test for the presence of small intestine or colon, e.g., in tissue sections, in a biopsy sample, in total RNA, etc. How to use polynucleotides in diagnostic assays is discussed below. In addition, the polynucleotides can serve as a target for therapy or drug discovery. A polypeptide, coded for by a selectively expressed polynucleotide, which is displayed on the cell-surface, can be a target for immunotherapy to treat, destroy, inhibit, etc., the diseased tissue. Selective transcripts can also be used in drug discovery schemes to identify pharmacological agents which suppress, inhibit, etc., their up-regulation, thereby preventing the phenotype associated with their expression. Thus, a selectively-expressed polynucleotide of the present invention has significant applications in diagnostic, therapeutic, prognostic, drug development, histology, pathology, and related areas.

A mammalian polynucleotide, or fragment thereof, of the present invention is a polynucleotide having a nucleotide sequence obtainable from a natural source. It therefore includes naturally-occurring normal, naturally-occurring mutant, and naturally-occurring polymorphic alleles (e.g., SNPs), differentially-spliced transcripts, splice-variants, etc. By the term “naturally-occurring,” it is meant that the polynucleotide is obtainable from a natural source, e.g., animal tissue and cells, body fluids, tissue culture cells, forensic samples. Natural sources include, e.g., living cells obtained from tissues and whole organisms, tumors, cultured cell lines, including primary and immortalized cell lines. Naturally-occurring mutations can include deletions (e.g., a truncated amino- or carboxy-terminus), substitutions, inversions, or additions of nucleotide sequence. These genes can be detected and isolated by polynucleotide hybridization according to methods which one skilled in the art would know, e.g., as discussed below.

A polynucleotide according to the present invention can be obtained from a variety of different sources. It can be obtained from DNA or RNA, such as polyadenylated mRNA or total RNA, e.g., isolated from tissues, cells, or whole organism. The polynucleotide can be obtained directly from DNA or RNA, from a cDNA library, from a genomic library, etc. The polynucleotide can be obtained from a cell or tissue (e.g., from an embryonic or adult tissues) at a particular stage of development, having a desired genotype, phenotype, disease status, etc.

The polynucleotides described in the SEQ ID NOS can be partial sequences that correspond to full-length, naturally-occurring transcripts. The present invention includes, as well, full-length polynucleotides that comprise these partial sequences, e.g., genomic DNAs and polynucleotides comprising a start and stop codon, a start codon and a polyA tail, a transcription start and a polyA tail, etc. These sequences can be obtained by any suitable method, e.g., using a partial sequence as a probe to select a fill-length cDNA from a library containing full-length inserts. A polynucleotide which “codes without interruption” refers to a polynucleotide having a continuous open reading frame (“ORF”) as compared to an ORF which is interrupted by introns or other noncoding sequences.

Polynucleotides and polypeptides can be excluded as compositions from the present invention if, e.g., listed in a publicly available databases on the day this application was filed and/or disclosed in a patent application having an earlier filing or priority date than this application and/or conceived and/or reduced to practice earlier than a polynucleotide in this application.

As described herein, the phrase “an isolated polynucleotide which is SEQ ID NO,” or “an isolated polynucleotide which is selected from SEQ ID NO,” refers to an isolated nucleic acid molecule from which the recited sequence was derived (e.g., a cDNA derived from mRNA; cDNA derived from genomic DNA). Because of sequencing errors, typographical errors, etc., the actual naturally-occurring sequence may differ from a SEQ ID listed herein. Thus, the phrase indicates the specific molecule from which the sequence was derived, rather than a molecule having that exact recited nucleotide sequence, analogously to how a culture depository number refers to a specific cloned fragment in a cryotube.

As explained in more detail below, a polynucleotide sequence of the invention can contain the complete sequence as shown in SEQ ID NOS 1-14 and 19-25, degenerate sequences thereof, anti-sense, muteins thereof, genes comprising said sequences, full-length cDNAs comprising said sequences, complete genomic sequences, fragments thereof, homologs, primers, nucleic acid molecules which hybridize thereto, derivatives thereof, etc.

Genomic

The present invention also relates genomic DNA from which the polynucleotides of the present invention can be derived. A genomic DNA coding for a human, mouse, or other mammalian polynucleotide, can be obtained routinely, for example, by screening a genomic library (e.g., a YAC library) with a polynucleotide of the present invention, or by searching nucleotide databases, such as GenBank and EMBL, for matches. Promoter and other regulatory regions can be identified upstream of coding and expressed RNAs, and assayed routinely for activity, e.g., by joining to a reporter gene (e.g., CAT, GFP, alkaline phosphatase, luciferase, galatosidase). A promoter obtained from a small intestine or colon selective gene can be used, e.g., in gene therapy to obtain tissue-specific expression of a heterologous gene (e.g., coding for a therapeutic product or cytotoxin).

Constructs

A polynucleotide of the present invention can comprise additional polynucleotide sequences, e.g., sequences to enhance expression, detection, uptake, cataloging, tagging, etc. A polynucleotide can include only coding sequence; a coding sequence and additional non-naturally occurring or heterologous coding sequence (e.g., sequences coding for leader, signal, secretory, targeting, enzymatic, fluorescent, antibiotic resistance, and other functional or diagnostic peptides); coding sequences and non-coding sequences, e.g., untranslated sequences at either a 5′ or 3′ end, or dispersed in the coding sequence, e.g., introns.

A polynucleotide according to the present invention also can comprise an expression control sequence operably linked to a polynucleotide as described above. The phrase “expression control sequence” means a polynucleotide sequence that regulates expression of a polypeptide coded for by a polynucleotide to which it is functionally (“operably”) linked. Expression can be regulated at the level of the mRNA or polypeptide. Thus, the expression control sequence includes mRNA-related elements and protein-related elements. Such elements include promoters, enhancers (viral or cellular), ribosome binding sequences, transcriptional terminators, etc. An expression control sequence is operably linked to a nucleotide coding sequence when the expression control sequence is positioned in such a manner to effect or achieve expression of the coding sequence. For example, when a promoter is operably linked 5′ to a coding sequence, expression of the coding sequence is driven by the promoter. Expression control sequences can include an initiation codon and additional nucleotides to place a partial nucleotide sequence of the present invention in-frame in order to produce a polypeptide (e.g., pET vectors from Promega have been designed to permit a molecule to be inserted into all three reading frames to identify the one that results in polypeptide expression). Expression control sequences can be heterologous or endogenous to the normal gene.

A polynucleotide of the present invention can also comprise nucleic acid vector sequences, e.g., for cloning, expression, amplification, selection, etc. Any effective vector can be used. A vector is, e.g., a polynucleotide molecule which can replicate autonomously in a host cell, e.g., containing an origin of replication. Vectors can be useful to perform manipulations, to propagate, and/or obtain large quantities of the recombinant molecule in a desired host. A skilled worker can select a vector depending on the purpose desired, e.g., to propagate the recombinant molecule in bacteria, yeast, insect, or mammalian cells. The following vectors are provided by way of example. Bacterial: pQE70, pQE60, pQE-9 (Qiagen), pBS, pD10, Phagescript, phiX174, pBK Phagemid, pNH8A, pNH16a, pNH18Z, pNH46A (Stratagene); Bluescript KS+II (Stratagene); ptrc99a, pKK223-3, pKK233-3, pDR54 0, pRIT5 (Pharmacia). Eukaryotic: PWLNEO, pSV2CAT, pOG44, pXT1, pSG (Stratagene), pSVK3, PBPV, PMSG, pSVL (Pharmacia), pCR2.1/TOPO, pCR11/TOPO, pCR4/TOPO, pTrcHisB, pCMV6-XL4, etc. However, any other vector, e.g., plasmids, viruses, or parts thereof may be used as long as they are replicable and viable in the desired host The vector can also comprise sequences which enable it to replicate in the host whose genome is to be modified.

Hybridization

Polynucleotide hybridization, as discussed in more detail below, is useful in a variety of applications, including, in gene detection methods, for identifying mutations, for making mutations, to identify homologs in the same and different species, to identify related members of the same gene family, in diagnostic and prognostic assays, in therapeutic applications (e.g., where an antisense polynucleotide is used to inhibit expression), etc.

The ability of two single-stranded polynucleotide preparations to hybridize together is a measure of their nucleotide sequence complementarity, e.g., base-pairing between nucleotides, such as A-T, G-C, etc. The invention thus also relates to polynucleotides, and their complements, which hybridize to a polynucleotide comprising a nucleotide sequence as set forth in SEQ ID NOS 1-14 and 19-25 and genomic sequences thereof. A nucleotide sequence hybridizing to the latter sequence will have a complementary polynucleotide strand, or act as a template for one in the presence of a polymerase (i.e., an appropriate polynucleotide synthesizing enzyme). The present invention includes both strands of polynucleotide, e.g., a sense strand and an anti-sense strand.

Hybridization conditions can be chosen to select polynucleotides which have a desired amount of nucleotide complementarity with the nucleotide sequences set forth in SEQ ID NOS 1-14 and 19-25 and genomic sequences thereof. A polynucleotide capable of hybridizing to such sequence, preferably, possesses, e.g., about 70%, 75%, 80%, 85%, 87%, 90%, 92%, 95%, 97%, 99%, or 100% complementarity, between the sequences. The present invention particularly relates to polynucleotide sequences which hybridize to the nucleotide sequences set forth in SEQ ID NOS 1-14 and 19-25 or genomic sequences thereof, under low or high stringency conditions. These conditions can be used, e.g., to select corresponding homologs in non-human species.

Polynucleotides which hybridize to polynucleotides of the present invention can be selected in various ways. Filter-type blots (i.e., matrices containing polynucleotide, such as nitrocellulose), glass chips, and other matrices and substrates comprising polynucleotides (short or long) of interest, can be incubated in a prehybridization solution (e.g., 6×SSC, 0.5% SDS, 100 μg/ml denatured salmon sperm DNA, 5× Denhardt's solution, and 50% formamide), at 22-68° C., overnight, and then hybridized with a detectable polynucleotide probe under conditions appropriate to achieve the desired stringency. In general, when high homology or sequence identity is desired, a high temperature can be used (e.g., 65° C.). As the homology drops, lower washing temperatures are used. For salt concentrations, the lower the salt concentration, the higher the stringency. The length of the probe is another consideration. Very short probes (e.g., less than 100 base pairs) are washed at lower temperatures, even if the homology is high. With short probes, formamide can be omitted. See, e.g., Current Protocols in Molecular Biology, Chapter 6, Screening of Recombinant Libraries; Sambrook et al., Molecular Cloning, 1989, Chapter 9.

For instance, high stringency conditions can be achieved by incubating the blot overnight (e.g., at least 12 hours) with a long polynucleotide probe in a hybridization solution containing, e.g., about 5×SSC, 0.5% SDS, 100 μg/ml denatured salmon sperm DNA and 50% formamide, at 42° C. Blots can be washed at high stringency conditions that allow, e.g., for less than 5% bp mismatch (e.g., wash twice in 0.1% SSC and 0.1% SDS for 30 min at 65° C.), i.e., selecting sequences having 95% or greater sequence identity.

Other non-limiting examples of high stringency conditions includes a final wash at 65° C. in aqueous buffer containing 30 mM NaCl and 0.5% SDS. Another example of high stringent conditions is hybridization in 7% SDS, 0.5 M NaPO₄, pH 7, 1 mM EDTA at 50° C., e.g., overnight, followed by one or more washes with a 1% SDS solution at 42° C. Whereas high stringency washes can allow for less than 5% mismatch, reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

Hybridization can also be based on a calculation of melting temperature (Tm) of the hybrid formed between the probe and its target, as described in Sambrook et al. Generally, the temperature Tm at which a short oligonucleotide (containing 18 nucleotides or fewer) will melt from its target sequence is given by the following equation: Tm=(number of A's and T's)×2° C.+(number of C's and G's)×4° C. For longer molecules, Tm=81.5+16.6 log₁₀[Na⁺]+0.41(% GC)-600/N where [Na⁺] is the molar concentration of sodium ions, % GC is the percentage of GC base pairs in the probe, and N is the length. Hybridization can be carried out at several degrees below this temperature to ensure that the probe and target can hybridize. Mismatches can be allowed for by lowering the temperature even further.

Stringent conditions can be selected to isolate sequences, and their complements, which have, e.g., at least about 90%, 95%, or 97%, nucleotide complementarity between the probe (e.g., a short polynucleotide of SEQ ID NOS 1-14 and 19-25 or genomic sequences thereof) and a target polynucleotide.

Other homologs of polynucleotides of the present invention can be obtained from mammalian and non-mammalian sources according to various methods. For example, hybridization with a polynucleotide can be employed to select homologs, e.g., as described in Sambrook et al., Molecular Cloning, Chapter 11, 1989. Such homologs can have varying amounts of nucleotide and amino acid sequence identity and similarity to such polynucleotides of the present invention. Mammalian organisms include, e.g., mice, rats, monkeys, pigs, cows, etc. Non-mammalian organisms include, e.g., vertebrates, invertebrates, zebra fish, chicken, Drosophila, C. elegans, Xenopus, yeast such as S. pombe, S. cerevisiae, roundworms, prokaryotes, plants, Arabidopsis, artemia, viruses, etc. The degree of nucleotide sequence identity between human and mouse can be about, e.g. 70% or more, 85% or more for open reading frames, etc.

Alignment

Alignments can be accomplished by using any effective algorithm. For pairwise alignments of DNA sequences, the methods described by Wilbur-Lipman (e.g., Wilbur and Lipman, Proc. Natl. Acad. Sci., 80:726-730, 1983) or Martinez/Needleman-Wunsch (e.g., Martinez, Nucleic Acid Res., 11:4629-4634, 1983) can be used. For instance, if the Martinez/Needleman-Wunsch DNA alignment is applied, the minimum match can be set at 9, gap penalty at 1.10, and gap length penalty at 0.33. The results can be calculated as a similarity index, equal to the sum of the matching residues divided by the sum of all residues and gap characters, and then multiplied by 100 to express as a percent. Similarity index for related genes at the nucleotide level in accordance with the present invention can be greater than 70%, 80%, 85%, 90%, 95%, 99%, or more. Pairs of protein sequences can be aligned by the Lipman-Pearson method (e.g., Lipman and Pearson, Science, 227:1435-1441, 1985) with k-tuple set at 2, gap penalty set at 4, and gap length penalty set at 12. Results can be expressed as percent similarity index, where related genes at the amino acid level in accordance with the present invention can be greater than 65%, 70%, 75%, 80%, 85%, 90%, 95%, 99%, or more. Various commercial and free sources of alignment programs are available, e.g., MegAlign by DNA Star, BLAST (National Center for Biotechnology Information), BCM (Baylor College of Medicine) Launcher, etc. After two sequences have been aligned, a “percent sequence identity” can be determined. For these purposes, it is convenient to refer to a Reference Sequence and a Compared Sequence, where the Compared Sequence is compared to the Reference Sequence. Percent sequence identity can be determined according to the following formula: Percent Identity=100 [1−(C/R)], wherein C is the number of differences between the Reference Sequence and the Compared Sequence over the length of alignment between the Reference Sequence and the Compared Sequence where (i) each base or amino acid in the Reference Sequence that does not have a corresponding aligned base or amino acid in the Compared Sequence, (ii) each gap in the Reference Sequence, (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

Percent sequence identity can also be determined by other conventional methods, e.g., as described in Altschul et al., Bull. Math. Bio. 48: 603-616, 1986 and Henikoff and Henikoff, Proc. Natl. Acad. Sci. USA 89:10915-10919, 1992.

Specific Polynucleotide Probes

A polynucleotide of the present invention can comprise any continuous nucleotide sequence of SEQ ID NOS 1-14 and 19-25, sequences which share sequence identity thereto, or complements thereof. The term “probe” refers to any substance that can be used to detect, identify, isolate, etc., another substance. A polynucleotide probe is comprised of nucleic acid can be used to detect, identify, etc., other nucleic acids, such as DNA and RNA.

These polynucleotides can be of any desired size that is effective to achieve the specificity desired. For example, a probe can be from about 7 or 8 nucleotides to several thousand nucleotides, depending upon its use and purpose. For instance, a probe used as a primer PCR can be shorter than a probe used in an ordered array of polynucleotide probes. Probe sizes vary, and the invention is not limited in any way by their size, e.g., probes can be from about 7-2000 nucleotides, 7-1000, 8-700, 8-600, 8-500, 8400, 8-300, 8-150, 8-100, 8-75, 7-50, 10-25, 14-16, at least about 8, at least about 10, at least about 15, at least about 25, etc. The polynucleotides can have non-naturally-occurring nucleotides, e.g., inosine, AZT, 3TC, etc. The polynucleotides can have 100% sequence identity or complementarity to a sequence of SEQ ID NOS 1-14 and 19-25, or it can have mismatches or nucleotide substitutions, e.g., 1, 2, 3, 4, or 5 substitutions. The probes can be single-stranded or double-stranded.

In accordance with the present invention, a polynucleotide can be present in a kit, where the kit includes, e.g., one or more polynucleotides, a desired buffer (e.g., phosphate, tris, etc.), detection compositions, RNA or cDNA from different tissues to be used as controls, libraries, etc. The polynucleotide can be labeled or unlabeled, with radioactive or non-radioactive labels as known in the art Kits can comprise one or more pairs of polynucleotides for amplifying nucleic acids specific for genes differentially expressed in small intestine or colon, e.g., comprising a forward and reverse primer effective in PCR. These include both sense and anti-sense orientations. For instance, in PCR-based methods (such as RT-PCR), a pair of primers are typically used, one having a sense sequence and the other having an antisense sequence.

Another aspect of the present invention is a nucleotide sequence that is specific to, or for, a selective polynucleotide. The phrases “specific for” or “specific to” a polynucleotide have a functional meaning that the polynucleotide can be used to identify the presence of one or more target genes in a sample. It is specific in the sense that it can be used to detect polynucleotides above background noise (“non-specific binding”). A specific sequence is a defined order of nucleotides which occurs in the polynucleotide, e.g., in the nucleotide sequences of SEQ ID NO 1-14 and 19-25. A probe or mixture of probes can comprise a sequence or sequences that are specific to a plurality of target sequences, e.g., where the sequence is a consensus sequence, a functional domain, etc., e.g., capable of recognizing a family of related genes. Such sequences can be used as probes in any of the methods described herein or incorporated by reference. Both sense and antisense nucleotide sequences are included. A specific polynucleotide according to the present invention can be determined routinely.

A polynucleotide comprising a specific sequence can be used as a hybridization probe to identify the presence of, e.g., human or mouse polynucleotide, in a sample comprising a mixture of polynucleotides, e.g., on a Northern blot. Hybridization can be performed under high stringent conditions (see, above) to select polynucleotides (and their complements which can contain the coding sequence) having at least 90%, 95%, 99%, etc., identity (i.e., complementarity) to the probe, but less stringent conditions can also be used. A specific polynucleotide sequence can also be fused in-frame, at either its 5′ or 3′ end, to various nucleotide sequences as mentioned throughout the patent, including coding sequences for enzymes, detectable markers, GFP, etc, expression control sequences, etc.

A polynucleotide probe, especially one that is specific to a polynucleotide of the present invention, can be used in gene detection and hybridization methods as already described. In one embodiment, a specific polynucleotide probe can be used to detect whether a particular tissue or cell-type is present in a target sample. To carry out such a method, a selective polynucleotide can be chosen which is characteristic of the desired target tissue. Such polynucleotide is preferably chosen so that it is expressed or displayed in the target tissue, but not in other tissues which are present in the sample. For instance, if detection of small intestine or colon is desired, it may not matter whether the selective polynucleotide is expressed in other tissues, as long as it is not expressed in cells normally present in blood, e.g., peripheral blood mononuclear cells. Starting from the selective polynucleotide, a specific polynucleotide probe can be designed which hybridizes (if hybridization is the basis of the assay) under the hybridization conditions to the selective polynucleotide, whereby the presence of the selective polynucleotide can be determined.

Probes which are specific for polynucleotides of the present invention can also be prepared using involve transcription-based systems, e.g., incorporating an RNA polymerase promoter into a selective polynucleotide of the present invention, and then transcribing anti-sense RNA using the polynucleotide as a template. See, e.g., U.S. Pat. No. 5,545,522.

Polynucleotide Composition

A polynucleotide according to the present invention can comprise, e.g., DNA, RNA, synthetic polynucleotide, peptide polynucleotide, modified nucleotides, dsDNA, ssDNA, ssRNA, dsRNA, and mixtures thereof. A polynucleotide can be single- or double-stranded, triplex, DNA:RNA, duplexes, comprise hairpins, and other secondary structures, etc. Nucleotides comprising a polynucleotide can be joined via various known linkages, e.g., ester, sulfamate, sulfamide, phosphorothioate, phosphoramidate, methylphosphonate, carbamate, etc., depending on the desired purpose, e.g., resistance to nucleases, such as RNAse H, improved in vivo stability, etc. See, e.g., U.S. Pat. No. 5,378,825. Any desired nucleotide or nucleotide analog can be incorporated, e.g., 6-mercaptoguanine, 8-oxo-guanine, etc.

Various modifications can be made to the polynucleotides, such as attaching detectable markers (avidin, biotin, radioactive elements, fluorescent tags and dyes, energy transfer labels, energy-emitting labels, binding partners, etc.) or moieties which improve hybridization, detection, and/or stability. The polynucleotides can also be attached to solid supports, e.g., nitrocellulose, magnetic or paramagnetic microspheres (e.g., as described in U.S. Pat. No. 5,411,863; U.S. Pat. No. 5,543,289; for instance, comprising ferromagnetic, supermagnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967, 5,476,925, and 5,478,893.

Polynucleotide according to the present invention can be labeled according to any desired method. The polynucleotide can be labeled using radioactive tracers such as ³²P, ³⁵S, ³H, or ¹⁴C, to mention some commonly used tracers. The radioactive labeling can be carried out according to any method, such as, for example, terminal labeling at the 3′ or 5′ end using a radiolabeled nucleotide, polynucleotide kinase (with or without dephosphorylation with a phosphatase) or a ligase (depending on the end to be labeled). A non-radioactive labeling can also be used, combining a polynucleotide of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

Nucleic Acid Detection Methods

Another aspect of the present invention relates to methods and processes for detecting small intestine or colon tissues. Detection methods have a variety of applications, including for diagnostic, prognostic, forensic, and research applications. To accomplish gene detection, a polynucleotide in accordance with the present invention can be used as a “probe.” The term “probe” or “polynucleotide probe” has its customary meaning in the art, e.g., a polynucleotide which is effective to identify (e.g., by hybridization), when used in an appropriate process, the presence of a target polynucleotide to which it is designed. Identification can involve simply determining presence or absence, or it can be quantitative, e.g., in assessing amounts of a gene or gene transcript present in a sample. Probes can be useful in a variety of ways, such as for diagnostic purposes, to identify homologs, and to detect, quantitate, or isolate a polynucleotide of the present invention in a test sample.

Assays can be utilized which permit quantification and/or presence/absence detection of a target nucleic acid in a sample. Assays can be performed at the single-cell level, or in a sample comprising many cells, where the assay is “averaging” expression over the entire collection of cells and tissue present in the sample. Any suitable assay format can be used, including, but not limited to, e.g., Southern blot analysis, Northern blot -analysis, polymerase chain reaction (“PCR”) (e.g., Saiki et al., Science, 241:53, 1988; U.S. Pat. Nos. 4,683,195, 4,683,202, and 6,040,166; PCR Protocols: A Guide to Methods and Applications, Innis et al., eds., Academic Press, New York, 1990), reverse transcriptase polymerase chain reaction (“RT-PCR”), anchored PCR, rapid amplification of cDNA ends (“RACE”) (e.g., Schaefer in Gene Cloning and Analysis: Current Innovations, Pages 99-115, 1997), ligase chain reaction (“LCR”) (EP 320 308), one-sided PCR (Ohara et al., Proc. Natl. Acad. Sci, 86:5673-5677, 1989), indexing methods (e.g., U.S. Pat. No. 5,508,169), in situ hybridization, differential display (e.g., Liang et al., Nucl. Acid. Res., 21:3269-3275, 1993; U.S. Pat. Nos. 5,262,311, 5,599,672 and 5,965,409; WO97/18454; Prashar and Weissman, Proc. Natl. Acad. Sci., 93:659-663, and U.S. Pat. Nos. 6,010,850 and 5,712,126; Welsh et al., Nucleic Acid Res., 20:4965-4970, 1992, and U.S. Pat. No. 5,487,985) and other RNA fingerprinting techniques, nucleic acid sequence based amplification (“NASBA”) and other transcription based amplification systems (e.g., U.S. Pat. Nos. 5,409,818 and 5,554,527; WO 88/10315), polynucleotide arrays (e.g., U.S. Pat. Nos. 5,143,854, 5,424,186; 5,700,637, 5,874,219, and 6,054,270; PCT WO 92/10092; PCT WO 90/15070), Qbeta Replicase (PCT/US87/00880), Strand Displacement Amplification (“SDA”), Repair Chain Reaction (“RCR”), nuclease protection assays, subtraction-based methods, Rapid-Scan™, etc. Additional useful methods include, but are not limited to, e.g., template-based amplification methods, competitive PCR (e.g., U.S. Pat. No. 5,747,251), redox-based assays (e.g., U.S. Pat. No. 5,871,918), Taqman-based assays (e.g., Holland et al., Proc. Natl. Acad, Sci., 88:7276-7280, 1991; U.S. Pat. Nos. 5,210,015 and 5,994,063), real-time fluorescence-based monitoring (e.g., U.S. Pat. No. 5,928,907), molecular energy transfer labels (e.g., U.S. Pat. Nos. 5,348,853, 5,532,129, 5,565,322, 6,030,787, and 6,117,635; Tyagi and Kramer, Nature Biotech., 14:303-309, 1996). Any method suitable for single cell analysis of gene or protein expression can be used, including in situ hybridization, immunocytochemistry, MACS, FACS, flow cytometry, etc. For single cell assays, expression products can be measured using antibodies, PCR, or other types of nucleic acid amplification (e.g., Brady et al., Methods Mol. & Cell. Biol. 2, 17-25, 1990; Eberwine et al., 1992, Proc. Natl. Acad. Sci., 89, 3010-3014, 1992; U.S. Pat. No. 5,723,290). These and other methods can be carried out conventionally, e.g., as described in the mentioned publications.

Many of such methods may require that the polynucleotide is labeled, or comprises a particular nucleotide type useful for detection. The present invention includes such modified polynucleotides that are necessary to carry out such methods. Thus, polynucleotides can be DNA, RNA, DNA:RNA hybrids, PNA, etc., and can comprise any modification or substituent which is effective to achieve detection.

Detection can be desirable for a variety of different purposes, including research, diagnostic, prognostic, and forensic. For diagnostic purposes, it may be desirable to identify the presence or quantity of a polynucleotide sequence in a sample, where the sample is obtained from tissue, cells, body fluids, etc. In a preferred method as described in more detail below, the present invention relates to a method of detecting a polynucleotide comprising, contacting a target polynucleotide in a test sample with a polynucleotide probe under conditions effective to achieve hybridization between the target and probe; and detecting hybridization.

Any test sample in which it is desired to identify a polynucleotide or polypeptide thereof can be used, including, e.g., blood, urine, saliva, stool (for extracting nucleic acid, see, e.g., U.S. Pat. No. 6,177,251), swabs comprising tissue, biopsied tissue, tissue sections, cultured cells, etc.

Detection can be accomplished in combination with polynucleotide probes for other genes, e.g., genes which are expressed in other disease states, tissues, cells, such as brain, heart, kidney, spleen, thymus, liver, stomach, small intestine, colon, muscle, lung, testis, placenta, pituitary, thyroid, skin, adrenal gland, pancreas, salivary gland, uterus, ovary, prostate gland, peripheral blood cells (T-cells, lymphocytes, etc.), embryo, normal breast fat, adult and embryonic stem cells, specific cell-types, such as endothelial, epithelial, myocytes, adipose, luminal epithelial, basoepithelial, myoepithelial, stromal cells, etc.

Polynucleotides can be used in wide range of methods and compositions, including for detecting, diagnosing, staging, grading, assessing, prognosticating, etc. diseases and disorders associated with SEQ ID NOS 1-14 and 19-25, for monitoring or assessing therapeutic and/or preventative measures, in ordered arrays, etc. Any method of detecting genes and polynucleotides of SEQ ID NOS 1-14 and 19-25 can be used; certainly, the present invention is not to be limited how such methods are implemented.

Along these lines, the present invention relates to methods of detecting small intestine or colon tissue in a sample comprising nucleic acid. Such methods can comprise one or more the following steps in any effective order, e.g., contacting said sample with a polynucleotide probe under conditions effective for said probe to hybridize specifically to nucleic acid in said sample, and detecting the presence or absence of probe hybridized to nucleic acid in said sample. Said probe can be a polynucleotide which is SEQ ID NOS 1-14 and 19-25, a polynucleotide having, e.g., about 70%, 80%, 85%, 90%, 95%, 99%, or more sequence identity thereto, effective or specific fragments thereof, or complements thereto. The detection method can be applied to any sample, e.g., cultured primary, secondary, or established cell lines, tissue biopsy, blood, urine, stool, and other bodily fluids, for any purpose.

Contacting the sample with probe can be carried out by any effective means in any effective environment. It can be accomplished in a solid, liquid, frozen, gaseous, amorphous, solidified, coagulated, colloid, etc., mixtures thereof, matrix. For instance, a probe in an aqueous medium can be contacted with a sample which is also in an aqueous medium, or which is affixed to a solid matrix, or vice-versa.

Generally, as used throughout the specification, the term “effective conditions” means, e.g., the particular milieu in which the desired effect is achieved. Such a milieu, includes, e.g., appropriate buffers, oxidizing agents, reducing agents, pH, co-factors, temperature, ion concentrations, suitable age and/or stage of cell (such as, in particular part of the cell cycle, or at a particular stage where particular genes are being expressed) where cells are being used, culture conditions (including substrate, oxygen, carbon dioxide, etc.). When hybridization is the chosen means of achieving detection, the probe and sample can be combined such that the resulting conditions are functional for said probe to hybridize specifically to nucleic acid in said sample.

The phrase “hybridize specifically” indicates that the hybridization between single-stranded polynucleotides is based on nucleotide sequence complementarity. The effective conditions are selected such that the probe hybridizes to a preselected and/or definite target nucleic acid in the sample. For instance, if detection of a polynucleotide set forth in SEQ ID NOS 1-14 and 19-25 is desired, a probe can be selected which can hybridize to such target gene under high stringent conditions, without significant hybridization to other genes in the sample. To detect homologs of a polynucleotide set forth in SEQ ID NOS 1-14 and 19-25, the effective hybridization conditions can be less stringent, and/or the probe can comprise codon degeneracy, such that a homolog is detected in the sample.

As already mentioned, the methods can be carried out by any effective process, e.g., by Northern blot analysis, polymerase chain reaction (PCR), reverse transcriptase PCR, RACE PCR, in situ hybridization, etc., as indicated above. When PCR based techniques are used, two or more probes are generally used. One probe can be specific for a defined sequence which is characteristic of a selective polynucleotide, but the other probe can be specific for the selective polynucleotide, or specific for a more general sequence, e.g., a sequence such as polyA which is characteristic of mRNA, a sequence which is specific for a promoter, ribosome binding site, or other transcriptional features, a consensus sequence (e.g., representing a functional domain). For the former aspects, 5′ and 3′ probes (e.g., polyA, Kozak, etc.) are preferred which are capable of specifically hybridizing to the ends of transcripts. When PCR is utilized, the probes can also be referred to as “primers” in that they can prime a DNA polymerase reaction.

In addition to testing for the presence or absence of polynucleotides, the present invention also relates to determining the amounts at which polynucleotides of the present invention are expressed in sample and determining the differential expression of such polynucleotides in samples. Such methods can involve substantially the same steps as described above for presence/absence detection, e.g., contacting with probe, hybridizing, and detecting hybridized probe, but using more quantitative methods and/or comparisons to standards.

The amount of hybridization between the probe and target can be determined by any suitable methods, e.g., PCR, RT-PCR, RACE PCR, Northern blot, polynucleotide microarrays, Rapid-Scan, etc., and includes both quantitative and qualitative measurements. For further details, see the hybridization methods described above and below. Determining by such hybridization whether the target is differentially expressed (e.g., up-regulated or down-regulated) in the sample can also be accomplished by any effective means. For instance, the target's expression pattern in the sample can be compared to its pattern in a known standard, such as in a normal tissue, or it can be compared to another gene in the same sample. When a second sample is utilized for the comparison, it can be a sample of normal tissue that is known not to contain diseased cells. The comparison can be performed on samples which contain the same amount of RNA (such as polyadenylated RNA or total RNA), or, on RNA extracted from the same amounts of starting tissue. Such a second sample can also be referred to as a control or standard. Hybridization can also be compared to a second target in the same tissue sample. Experiments can be performed that determine a ratio between the target nucleic acid and a second nucleic acid (a standard or control), e.g., in a normal tissue. When the ratio between the target and control are substantially the same in a normal and sample, the sample is determined or diagnosed not to contain cells. However, if the ratio is different between the normal and sample tissues, the sample is determined to contain cancer cells. The approaches can be combined, and one or more second samples, or second targets can be used. Any second target nucleic acid can be used as a comparison, including “housekeeping” genes, such as beta-actin, alcohol dehydrogenase, or any other gene whose expression does not vary depending upon the disease status of the cell.

A goal, among others, of the method is to determine (i.e., identify) the presence of kidney tissue or cells in a sample of any origin. This can be accomplished by deciding whether one or more genes in a set of target genes are expressed in the sample of interest. Although the genes are selectively expressed in small intestine or colon, because of variability between individuals and tissue samples, each gene may not be expressed 100% of the time in small intestine or colon. There are many sources of variability that account for differences in gene penetrance, including, the state of the tissue and cells (e.g., normal, inflamed, diseased), cell cycle status, effects of other genes, environmental effects, age, health, gender, etc. Additionally, a selectively expressed gene may also be expressed in other tissue types. For instance, a selectively expressed gene can be expressed in multiple tissues, e.g., small intestine and brain. Thus, expression of it in a sample indicates that the tissue is more likely to be either small intestine or brain, than another tissue type, but this one probe is insufficient to distinguish between the two. For certain purposes, this level of certainty may be adequate. Determining that a second selectively nucleotide sequence for small intestine or colon is expressed in the sample provides greater certainty that the sample is small intestine or colon, not brain. For these and other reasons, certainty or probability that a given sample is small intestine or colon can be correlated with the number of selective genes expressed in the sample. Successive probes can be chosen based on their specificities. A greater number of genes determined to be expressed in a sample can indicate that there is a higher probability that the sample comprises small intestine or colon tissue. Probability values can be determined statistically and/or empirically, e.g., by making many measurements on individuals in a given population and determining the frequency in which the gene is expressed. These values can differ, depending upon the selected population, e.g., gender, health, ancestry, age, etc.

By the phrase “target genes,” it is meant the genes that the method is aimed at determining. Each of the nucleotide sequences shown in SEQ ID NOS 1-14 and 19-25 represents a region of a target gene, i.e., a fragment of a complete gene (e.g., a gene has regulatory and coding sequences) serving as a specific identification label for that target gene. The expression of the genes in a sample can be determined by any effective method. The term “expression” means, e.g., transcription of the gene into RNA, or translation of an RNA into protein. Expression can be determined, e.g., by detecting RNA, by detecting polypeptide translated from the RNA, or any product produced during expression of the gene. Nucleic acid and polypeptide detection are routine, and can be accomplished as described herein or as the skilled worker would know. For example, detecting of RNA can be performed by Northern blot analysis, polymerase chain reaction (PCR), reverse transcriptase PCR, RACE PCR, or in situ hybridization using a polynucleotide probe which is SEQ ID NOS 1-14 and 19-25, a polynucleotide having sequence identity thereto, effective specific fragments thereof, complements thereto, and said polynucleotide is selectively expressed in said kidney. Any amount of sequence identity is suitable as long as it maintains the desired amount of specificity.

Methods of Identifying Polymorphisms, Mutations, etc.

Polynucleotides of the present invention can also be utilized to identify mutant alleles, SNPs, gene rearrangements and modifications, and other polymorphisms of the wild-type gene. Mutant alleles, polymorphisms, SNPs, etc., can be identified and isolated from cancers that are known, or suspected to have, a genetic component. Identification of such genes can be carried out routinely (see, above for more guidance), e.g., using PCR, hybridization techniques, direct sequencing, mismatch reactions (see, e.g., above), RFLP analysis, SSCP (e.g., Orita et al., Proc. Natl. Acad. Sci., 86:2766, 1992), etc., where a polynucleotide having a sequence selected from SEQ ID NOS 1-14 and 19-25 is used as a probe. Examples of useful fragments are shown in SEQ ID NOS 1-14 and 19-25. The selected mutant alleles, SNPs, polymorphisms, etc., can be used diagnostically to determine whether a subject has, or is susceptible to a disorder associated with SEQ ID NOS 1-14 and 19-25, as well as to design therapies and predict the outcome of the disorder. Methods involve, e.g., diagnosing a disorder associated with SEQ ID NOS 1-14 and 19-25, comprising, detecting the presence of a mutation in a gene represented by a polynucleotide selected from SEQ ID NOS 1-14 and 19-25. The detecting can be carried out by any effective method, e.g., obtaining cells from a subject, determining the gene sequence or structure of a target gene (using, e.g., mRNA, cDNA, genomic DNA, etc), comparing the sequence or structure of the target gene to the structure of the normal gene, whereby a difference in sequence or structure indicates a mutation in the gene in the subject. Polynucleotides can also be used to test for mutations, SNPs, polymorphisms, etc., e.g., using mismatch DNA repair technology as described in U.S. Pat. No. 5,683,877; U.S. Pat. No. 5,656,430; Wu et al., Proc. Natl. Acad. Sci., 89:8779-8783, 1992.

The present invention also relates to methods of detecting polymorphisms in SEQ ID NOS 1-14 and 19-25, comprising, e.g., comparing the structure of: genomic DNA comprising all or part of SEQ ID NOS 1-14 and 19-25, mRNA comprising all or part of SEQ ID NOS 1-14 and 19-25, cDNA comprising all or part of SEQ ID NOS 1-14 and 19-25, or a polypeptide comprising all or part of SEQ ID NOS 1-14 and 19-25 (e.g., any of the polypeptide sequences set forth among SEQ ID NOS 1-14 and 19-25], with the structure of SEQ ID NOS 1-14 and 19-25. The methods can be carried out on a sample from any source, e.g., cells, tissues, body fluids, blood, urine, stool, hair, egg, sperm, etc.

These methods can be implemented in many different ways. For example, “comparing the structure” steps include, but are not limited to, comparing restriction maps, nucleotide sequences, amino acid sequences, RFLPs, Dnase sites, DNA methylation fingerprints (e.g., U.S. Pat. No. 6,214,556), protein cleavage sites, molecular weights, electrophoretic mobilities, charges, ion mobility, etc., between a standard SEQ ID NOS 1-14 and 19-25 and a test sequence. The term “structure” can refer to any physical characteristics or configurations which can be used to distinguish between nucleic acids and polypeptides. The methods and instruments used to accomplish the comparing step depends upon the physical characteristics which are to be compared. Thus, various techniques are contemplated, including, e.g., sequencing machines (both amino acid and polynucleotide), electrophoresis, mass spectrometer (U.S. Pat. Nos. 6,093,541, 6,002,127), liquid chromatography, HPLC, etc.

To carry out such methods, “all or part” of the gene or polypeptide can be compared. For example, if nucleotide sequencing is utilized, the entire gene can be sequenced, including promoter, introns, and exons, or only parts of it can be sequenced and compared, e.g., exon 1, exon 2, etc.

Mutagenesis

Mutated polynucleotide sequences of the present invention are useful for various purposes, e.g., to create mutations of the polypeptides they encode, to identify functional regions of genomic DNA, to produce probes for screening libraries, etc. Mutagenesis can be carried out routinely according to any effective method, e.g., oligonucleotide-directed (Smith, M., Ann. Rev. Genet. 19:423463, 1985), degenerate oligonucleotide-directed (Hill et al., Method Enzymology, 155:558-568, 1987), region-specific (Myers et al., Science, 229:242-246, 1985; Derbyshire et al., Gene, 46:145, 1986; Ner et al., DNA, 7:127, 1988), tinker-scanning (McKnight and Kingsbury, Science, 217:316-324, 1982), directed using PCR, recursive ensemble mutagenesis (Arliin and Yourvan, Proc. Natl. Acad. Sci., 89:7811-7815, 1992), random mutagenesis (e.g., U.S. Pat. Nos. 5,096,815; 5,198,346; and 5,223,409), site-directed mutagenesis (e.g., Walder et al., Gene, 42:133, 1986; Bauer et al., Gene, 37:73, 1985; Craik, Bio Techniques, January 1985, 12-19; Smith et al., Genetic Engineering: Principles and Methods, Plenum Press, 1981), phage display (e.g., Lowman et al., Biochem. 30:10832-10837, 1991; Ladner et al., U.S. Pat. No. 5,223,409; Huse, WIPO Publication WO 92/06204), etc. Desired sequences can also be produced by the assembly of target sequences using mutually priming oligonucleotides (Uhlmann, Gene, 71:29-40, 1988). For directed mutagenesis methods, analysis of the three-dimensional structure of any of the polypeptides, e.g., shown in SEQ ID NOS 1-14 and 19-25, can be used to guide and facilitate making mutants which effect polypeptide activity. Sites of substrate-enzyme interaction or other biological activities can also be determined by analysis of crystal structure as determined by such techniques as nuclear magnetic resonance, crystallography or photoaffinity labeling. See, for example, de Vos et al., Science 255:306-312, 1992; Smith et al., J. Mol. Biol. 224:899-904, 1992; Wlodaver et al., FEBS Lett. 309:59-64, 1992.

In addition, libraries of SEQ ID NOS 1-14 and 19-25 and fragments thereof can be used for screening and selection of SEQ ID NOS 1-14 and 19-25 variants. For instance, a library of coding sequences can be generated by treating a double-stranded DNA with a nuclease under conditions where the nicking occurs, e.g., only once per molecule, denaturing the double-stranded DNA, renaturing it to for double-stranded DNA that can include sense/antisense pairs from different nicked products, removing single-stranded portions from reformed duplexes by treatment with S1 nuclease, and ligating the resulting DNAs into an expression vector. By this method, expression libraries can be made comprising “mutagenized” SEQ ID NOS 1-14 and 19-25. The entire coding sequence or parts thereof can be used.

Polynucleotide Expression, Polypeptides Produced Thereby, and Specific-Binding Partners Thereto.

A polynucleotide according to the present invention can be expressed in a variety of different systems, in vitro and in vivo, according to the desired purpose. For example, a polynucleotide can be inserted into an expression vector, introduced into a desired host, and cultured under conditions effective to achieve expression of a polypeptide coded for by the polynucleotide, to search for specific binding partners. Effective conditions include any culture conditions which are suitable for achieving production of the polypeptide by the host cell, including effective temperatures, pH, medium, additives to the media in which the host cell is cultured (e.g., additives which amplify or induce expression such as butyrate, or methotrexate if the coding polynucleotide is adjacent to a dhfr gene), cycloheximide, cell densities, culture dishes, etc. A polynucleotide can be introduced into the cell by any effective method including, e.g., naked DNA, calcium phosphate precipitation, electroporation, injection, DEAE-Dextran mediated transfection, fusion with liposomes, association with agents which enhance its uptake into cells, viral transfection. A cell into which a polynucleotide of the present invention has been introduced is a transformed host cell. The polynucleotide can be extrachromosomal or integrated into a chromosome(s) of the host cell. It can be stable or transient. An expression vector is selected for its compatibility with the host cell. Host cells include, mammalian cells, e.g., COS, CV1, BHK, CHO, HeLa, LTK, NIH 3T3, FHs, 74 Int (ATCC CCL-241), Hs 1.Int (ATCC CRL7820), Hs 738.St/Int (ATCC CRL-7869), IEC-6 (ATCC CRL-1592), IA-XsSBR (ATCC CRL-1677), FC114 E.Int (ATCC CRL-6166), WiDr (ATCC CCL-218), COLO 320DM (ATCC CCL-220), HCT-15 (ATCC CCL-225), SW620 (ATCC CCL-227), LoVo (ATCC CCL-229), HCT-8 (ATCC CCL-244), T84 (ATCC CCL-248), NCI-H548 (ATCC CCL-249), LS123 (ATCC CCL-255), CCD-18Co (ATCC CRL-1459), FHC (ATCC CRL-1831), Hs 207.T (ATCC CRL-7168), SNU-C1 (ATCC CRL-5972), Caco-2 (ATCC HTB-37), HT-29, SK-CO-1 (ATCC HT-39), Hs 257.T (ATCC CRL-7214), and other small intestine and colon primary and established cell lines, insect cells, such as Sf9 (S. frugipeda) and Drosophila, bacteria, such as E. coli, Streptococcus, bacillus, yeast, such as Sacharomyces, S. cerevisiae, fungal cells, plant cells, embryonic or adult stem cells (e.g., mammalian, such as mouse or human).

Expression control sequences are similarly selected for host compatibility and a desired purpose, e.g., high copy number, high amounts, induction, amplification, controlled expression. Other sequences which can be employed include enhancers such as from SV40, CMV, RSV, inducible promoters, cell-type specific elements, or sequences which allow selective or specific cell expression. Promoters that can be used to drive its expression, include, e.g., the endogenous promoter, MMTV, SV40, trp, lac, tac, or T7 promoters for bacterial hosts; or alpha factor, alcohol oxidase, or PGH promoters for yeast. RNA promoters can be used to produced RNA transcripts, such as T7 or SP6. See, e.g., Melton et al., Polynucleotide Res., 12(18):7035-7056, 1984; Dunn and Studier. J. Mol. Bio., 166:477-435, 1984; U.S. Pat. No. 5,891,636; Studier et al., Gene Expression Technology, Methods in Enzymology, 85:60-89, 1987. In addition, as discussed above, translational signals (including in-frame insertions) can be included.

When a polynucleotide is expressed as a heterologous gene in a transfected cell line, the gene is introduced into a cell as described above, under effective conditions in which the gene is expressed. The term “heterologous” means that the gene has been introduced into the cell line by the “hand-of-man.” Introduction of a gene into a cell line is discussed above. The transfected (or transformed) cell expressing the gene can be lysed or the cell line can be used intact.

For expression and other purposes, a polynucleotide can contain codons found in a naturally-occurring gene, transcript, or cDNA, for example, e.g., as set forth in SEQ ID NOS 1-14 and 19-25, or it can contain degenerate codons coding for the same amino acid sequences. For instance, it may be desirable to change the codons in the sequence to optimize the sequence for expression in a desired host. See, e.g., U.S. Pat. Nos. 5,567,600 and 5,567,862.

A polypeptide according to the present invention can be recovered from natural sources, transformed host cells (culture medium or cells) according to the usual methods, including, detergent extraction (e.g., non-ionic detergent, Triton X-100, CHAPS, octylglucoside, Igepal CA-630), ammonium sulfate or ethanol precipitation, acid extraction, anion or cation exchange chromatography, phosphocellulose chromatography, hydrophobic interaction chromatography, hydroxyapatite chromatography, lectin chromatography, gel electrophoresis. Protein refolding steps can be used, as necessary, in completing the configuration of the mature protein. Finally, high performance liquid chromatography (HPLC) can be employed for purification steps. Another approach is express the polypeptide recombinantly with an affinity tag (Flag epitope, HA epitope, myc epitope, 6×His, maltose binding protein, chitinase, etc) and then purify by anti-tag antibody-conjugated affinity chromatography.

The present invention also relates to antibodies, and other specific-binding partners, which are specific for polypeptides encoded by polynucleotides of the present invention, e.g., SEQ ID NOS 1-14 and 19-25. Antibodies, e.g., polyclonal, monoclonal, recombinant, chimeric, humanized, single-chain, Fab, and fragments thereof, can be prepared according to any desired method. See, also, screening recombinant immunoglobulin libraries (e.g., Orlandi et al., Proc. Natl. Acad. Sci., 86:3833-3837, 1989; Huse et al., Science, 256:1275-1281, 1989); in vitro stimulation of lymphocyte populations; Winter and Milstein, Nature, 349: 293-299, 1991. The antibodies can be IgM, IgG, subtypes, IgG2a, IgG1, etc. Antibodies, and immune responses, can also be generated by administering naked DNA See, e.g., U.S. Pat. Nos. 5,703,055; 5,589,466; 5,580,859. Antibodies can be used from any source, including, goat, rabbit, mouse, chicken (e.g., IgY; see, Duan, W0/029444 for methods of making antibodies in avian hosts, and harvesting the antibodies from the eggs). An antibody specific for a polypeptide means that the antibody recognizes a defined sequence of amino acids within or including the polypeptide. Other specific binding partners include, e.g., aptamers and PNA. antibodies can be prepared against specific epitopes or domains of SEQ ID NOS 1-14 and 19-25,.

The preparation of polyclonal antibodies is well-known to those skilled in the art. See, for example, Green et al., Production of Polyclonal Antisera, in IMMUNOCHEMICAL PROTOCOLS (Manson, ed.), pages 1-5 (Humana Press 1992); Coligan et al., Production of Polyclonal Antisera in Rabbits, Rats, Mice and Hamsters, in CURRENT PROTOCOLS IN IMMUNOLOGY, section 2.4.1 (1992). The preparation of monoclonal antibodies likewise is conventional. See, for example, Kohler & Milstein, Nature 256:495 (1975); Coligan et al., sections 2.5.1-2.6.7; and Harlow et al., ANTIBODIES: A LABORATORY MANUAL, page 726 (Cold Spring Harbor Pub. 1988).

Antibodies can also be humanized, e.g., where they are to be used therapeutically. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Natl. Acad. Sci. USA 86:3833 (1989), which is hereby incorporated in its entirety by reference. Techniques for producing humanized monoclonal antibodies are described, for example, in U.S. Pat. No. 6,054,297, Jones et al., Nature 321: 522 (1986); Riechmann et al., Nature 332: 323 (1988); Verhoeyen et al., Science 239: 1534 (1988); Carter et al., Proc. Nat'l Acad. Sci. USA 89: 4285 (1992); Sandhu, Crit. Rev. Biotech. 12: 437 (1992); and Singer et al., J. Immunol. 150: 2844 (1993).

Antibodies of the invention also may be derived from human antibody fragments isolated from a combinatorial immunoglobulin library. See, for example, Barbas et al., METHODS: A COMPANION TO METHODS IN ENZYMOLOGY, VOL. 2, page 119 (1991); Winter et al., Ann. Rev. Immunol. 12: 433 (1994). Cloning and expression vectors that are useful for producing a human immunoglobulin phage library can be obtained commercially, for example, from STRATAGENE Cloning Systems (La Jolla, Calif.).

In addition, antibodies of the present invention may be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the, endogenous heavy and light chain loci. The transgenic mice can synthesize human antibodies specific for human antigens and can be used to produce human antibody-secreting hybridomas. Methods for obtaining human antibodies from transgenic mice are described, e.g., in Green et al., Nature Genet. 7:13 (1994); Lonberg et al., Nature 368:856 (1994); and Taylor et al., Int. Immunol. 6:579 (1994).

Antibody fragments of the present invention can be prepared by proteolytic hydrolysis of the antibody or by expression in E. coli of nucleic acid encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′).sub.2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. No. 4,036,945 and No. 4,331,647, and references contained therein. These patents are hereby incorporated in their entireties by reference. See also Nisoiihoff et al., Arch. Biochem. Biophys. 89:230 (1960); Porter, Biochem. J. 73:119 (1959); Edelman et al, METHODS IN ENZYMOLOGY, VOL. 1, page 422 (Academic Press 1967); and Coligan et al. at sections 2.8.1-2.8.10 and 2.10.1-2.10.4.

Other methods of cleaving antibodies, such as separation of heavy chains to form monovalent light-heavy chain fragments, further cleavage of fragments, or other enzymatic, chemical, or genetic techniques can also be used. For example, Fv fragments comprise an association of V.sub.H and V.sub.L chains. This association may be noncovalent, as described in Inbar et al., Proc. Nat'l Acad. Sci. USA 69:2659 (1972). Alternatively, the variable chains can be linked by an intermolecular disulfide bond or cross-linked by chemicals such as glutaraldehyde. See, e.g., Sandhu, supra. Preferably, the Fv fragments comprise V.sub.H and V.sub.L chains connected by a peptide linker. These single-chain antigen binding proteins (sFv) are prepared by constructing a structural gene comprising nucleic acid sequences encoding the V.sub.H and V.sub.L domains connected by an oligonucleotide. The structural gene is inserted into an expression vector, which is subsequently introduced into a host cell such as E. coli. The recombinant host, cells synthesize a single polypeptide chain with a linker peptide bridging the two V domains. Methods for producing sFvs are described, for example, by Whitlow et al., METHODS: A COMPANION TO METHODS IN ENZYMOLOGY, VOL. 2, page 97 (1991); Bird et al., Science 242:423-426 (1988); Ladneret al., U.S. Pat. No. 4,946,778; Pack et al., Bio/Technology 11: 1271-77 (1993); and Sandhu, supra.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick et al., METHODS: A COMPANION TO METHODS IN ENZYMOLOGY, VOL. 2, page 106 (1991).

The term “antibody” as used herein includes intact molecules as well as fragments thereof, such as Fab, F(ab′)2, and Fv which are capable of binding to an epitopic determinant present in Bin1 polypeptide. Such antibody fragments retain some ability to selectively bind with its antigen or receptor. The term “epitope” refers to an antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics. Antibodies can be prepared against specific epitopes or polypeptide domains.

Antibodies which bind to SEQ ID NOS 1-14 and 19-25 polypeptides of the present invention can be prepared using an intact polypeptide or fragments containing small peptides of interest as the immunizing antigen. For example, it may be desirable to produce antibodies that specifically bind to the N- or C-terminal domains of SEQ ID NOS 1-14 and 19-25. The polypeptide or peptide used to immunize an animal which is derived from translated cDNA or chemically synthesized which can be conjugated to a carrier protein, if desired. Such commonly used carriers which are chemically coupled to the immunizing peptide include keyhole limpet hemocyanin (KLH), thyroglobulin, bovine serum albumin (BSA), and tetanus toxoid.

Polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (See for example, Coligan, et al., Unit 9, Current Protocols in Immunology, Wiley Interscience, 1994, incorporated by reference).

Anti-idiotype technology can also be used to produce invention monoclonal antibodies which mimic an epitope. For example, an anti-idiotypic monoclonal antibody made to a first monoclonal antibody will have a binding domain in the hypervariable region which is the “image” of the epitope bound by the first monoclonal antibody.

Methods of Detecting Polypeptides

Polypeptides coded for by SEQ ID NOS 1-14 and 19-25 of the present invention can be detected, visualized, determined, quantitated, etc. according to any effective method. useful methods include, e.g., but are not limited to, immunoassays, RIA (radioimmunassay), ELISA, (enzyme-linked-immunosorbent assay), immunoflourescence, flow cytometry, histology, electron microscopy, light microscopy, in situ assays, immunoprecipitation, Western blot, etc.

Immunoassays may be carried in liquid or on biological support. For instance, a sample (e.g., blood, stool, urine, cells, tissue, body fluids, etc.) can be brought in contact with and immobilized onto a solid phase support or carrier such as nitrocellulose, or other solid support that is capable of immobilizing cells, cell particles or soluble proteins. The support may then be washed with suitable buffers followed by treatment with the detectably labeled SEQ ID NOS 1-14 and 19-25 specific antibody. The solid phase support can then be washed with a buffer a second time to remove unbound antibody. The amount of bound label on solid support may then be detected by conventional means.

A “solid phase support or carrier” includes any support capable of binding an antigen, antibody, or other specific binding partner. Supports or carriers include glass, polystyrene, polypropylene, polyethylene, dextran, nylon, amylases, natural and modified celluloses, polyacrylamides, and magnetite. A support material can have any structural or physical configuration. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads

One of the many ways in which gene peptide-specific antibody can be detectably labeled is by linking it to an enzyme and using it in an enzyme immunoassay (EIA). See, e.g., Voller, A., “The Enzyme Linked Immunosorbent Assay (ELISA),” 1978, Diagnostic Horizons 2, 1-7, Microbiological Associates Quarterly Publication, Walkersville, Md.); Voller, A. et al., 1978, J. Clin. Pathol. 31, 507-520; Butler, J. E., 1981, Meth. Enzymol. 73, 482-523; Maggio, E. (ed.), 1980, Enzyme Immunoassay, CRC Press, Boca Raton, Fla. The enzyme which is bound to the antibody will react with an appropriate substrate, preferably a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, .alpha.-glycerophosphate, dehydrogenase, triose phosphate isomerase, horseradish peroxidase, alkaline phosphatase, asparaginase, glucose oxidase, beta.-galactosidase, ribonuclease, urease, catalase, glucose-6-phosphate dehydrogenase, glucoamylase and acetylcholinesterase. The detection can be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect SEQ ID NOS 1-14 and 19-25 peptides through the use of a radioimmunoassay (RIA). See, e.g., Weintraub, B., Principles of Radioimmunoassays, Seventh Training Course on Radioligand Assay Techniques, The Endocrine Society, March, 1986. The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The antibody can also be detectably labeled using fluorescence emitting metals such as those in the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Diagnostic

The present invention also relates to methods and compositions for diagnosing a small intestine or colon disorder using polynucleotides, polypeptides, and specific-binding partners of the present invention to detect, assess, determine, etc., SEQ ID NOS 1-14 and 19-25. In such methods, the gene can serve as a marker for the disorder, e.g., where the gene, when mutant, is a direct cause of the disorder; where the gene is affected by another gene(s) which is directly responsible for the disorder, e.g., when the gene is part of the same signaling pathway as the directly responsible gene; and, where the gene is chromosomally linked to the gene(s) directly responsible for the disorder, and segregates with it. Many other situations are possible. To detect, assess, determine, etc., a probe specific for the gene can be employed as described above and below. Any method of detecting and/or assessing the gene can be used, including detecting expression of the gene using polynucleotides, antibodies, or other specific-binding partners.

The present invention relates to methods of diagnosing a small intestine or colon disorders, comprising, e.g., assessing the expression of SEQ ID NOS 1-14 and 19-25 in a tissue sample comprising tissue or cells suspected of having the disorder (e.g., where the sample comprises small intestine or colon). The phrase “diagnosing” indicates that it is determined whether the sample has the disorder. A “disorder” means, e.g., any abnormal condition as in a disease or malady.

By the phrase “assessing expression of SEQ ID NOS 1-14 and 19-25,” it is meant that the functional status of the gene is evaluated. This includes, but is not limited to, measuring expression levels of said gene, determining the genomic structure of said gene, determining the mRNA structure of transcripts from said gene, or measuring the expression levels of polypeptide coded for by said gene. Thus, the term “assessing expression” includes evaluating the all aspects of the transcriptional and translational machinery of the gene. For instance, if a promoter defect causes, or is suspected of causing, the disorder, then a sample can be evaluated (i.e., “assessed”) by looking (e.g., sequencing or restriction mapping) at the promoter sequence in the gene, by detecting transcription products (e.g., RNA), by detecting translation product (e.g., polypeptide). Any measure of whether the gene is functional can be used, including, polypeptide, polynucleotide, and functional assays for the gene's biological activity.

In making the assessment, it can be useful to compare the results to a normal gene, e.g., a gene which is not associated with the disorder. The nature of the comparison can be determined routinely, depending upon how the assessing is accomplished. If, for example, the mRNA levels of a sample is detected, then the mRNA levels of a normal can serve as a comparison, or a gene which is known not to be affected by the disorder. Methods of detecting mRNA are well known, and discussed above, e.g., but not limited to, Northern blot analysis, polymerase chain reaction (PCR), reverse transcriptase PCR, RACE PCR, etc. Similarly, if polypeptide production is used to evaluate the gene, then the polypeptide in a normal tissue sample can be used as a comparison, or, polypeptide from a different gene whose expression is known not to be affected by the disorder. These are only examples of how such a method could be carried out.

Assessing the effects of therapeutic and preventative interventions (e.g., administration of a drug, chemotherapy, radiation, etc.) on small intestine and colon disorders is a major effort in drug discovery, clinical medicine, and pharmacogenomics. The evaluation of therapeutic and preventative measures, whether experimental or already in clinical use, has broad applicability, e.g., in clinical trials, for monitoring the status of a patient, for analyzing and assessing animal models, and in any scenario involving cancer treatment and prevention. Analyzing the expression profiles of polynucleotides of the present invention can be utilized as a parameter by which interventions are judged and measured. Treatment of a disorder can change the expression profile in some manner which is prognostic or indicative of the drug's effect on it. Changes in the profile can indicate, e.g., drug toxicity, return to a normal level, etc. Accordingly, the present invention also relates to methods of monitoring or assessing a therapeutic or preventative measure (e.g., chemotherapy, radiation, anti-neoplastic drugs, antibodies, etc.) in a subject having a small intestine or colon disorder, or, susceptible to such a disorder, comprising, e.g., detecting the expression levels of SEQ ID NOS 1-14 and 19-25. A subject can be a cell-based assay system, non-human animal model, human patient, etc. Detecting can be accomplished as described for the methods above and below. By “therapeutic or preventative intervention,” it is meant, e.g., a drug administered to a patient, surgery, radiation, chemotherapy, and other measures taken to prevent, treat, or diagnose a disorder.

Polynucleotides of the present invention can be used to identify, detect, stage, determine the presence of, prognosticate, treat, study, etc., diseases and conditions of the small intestine. These include, but are not limited to, Crohn's disease, colitis, inflammatory bowel disease, tumors, benign tumors, such as benign stromal tumors, adenoma, angioma, adenomatous (pedunculated and sessile) polyps, malignant, carcinoid tumors, endocrine cell tumors, lymphoma, adenocarcinoma, foregut, midgut, and hindgut carcinoma, gastroinstestinal stromal tumor (GIST), such as leiomyoma, cellular leiomyoma, leiomyoblastoma, and leiomyosarcoma, gastrointestinal autonomic nerve tumor, malabsorption syndromes, celiac diseases, diverticulosis, Meckel's diverticulum, colonic diverticula, megacolon, Hirschsprung's disease, irritable bowel syndrome, mesenteric ischemia, ischemic colitis, colorectal cancer, colonic polyposis, polyp syndrome, etc.

Polynucleotides can also be used for staging and classifying conditions and diseases of the present invention, alone, or in combination with conventional staging and classification schemes.

As discussed elsewhere, expression can be assessed in any sample comprising any tissue or cell type, body fluid, etc., including cells from small intestine can be used, or cells derived from small intestine or colon. By the phrase “cells derived from small intestine or colon,” it is meant that the derived cells originate from small intestine or colon, e.g., when metastasis from a primary tumor site has occurred, when a progenitor-type or pluripotent cell gives rise to other cells, etc.

Identifying Agent Methods

The present invention also relates to methods of identifying agents that modulate the expression of SEQ ID NOS 1-14 and 19-25 expressed in small intestine or colon cells, comprising, in any effective order, one or more of the following steps, e.g., contacting a cell population with a test agent under conditions effective for said test agent to modulate the expression of SEQ ID NOS 1-14 and 19-25 in said cell population, and determining whether said test agent modulates said SEQ ID NOS 1-14 and 19-25. An agent can modulate expression of SEQ ID NOS 1-14 and 19-25 at any level, including transcription, translation, and/or perdurance of the nucleic acid or polypeptide (e.g., degradation, stability, etc.) product in the cell.

Contacting the cell population with the test agent can be accomplished by any suitable method and/or means that places the agent in a position to functionally control expression of the SEQ ID NOS 1-14 and 19-25 present in cells within the population. Functional control indicates that the agent can exert its physiological effect on the cell through whatever mechanism it works. The choice of the method and/or means can depend upon the nature of the agent and the condition and type of the cell population (such as, in vivo, in vitro, organ explants, etc.). For instance, if the cell population is an in vitro cell culture, the agent can be contacted with the cells by adding it directly into the culture medium. If the agent cannot dissolve readily in an aqueous medium, it can be incorporated into liposomes, or another lipophilic carrier, and then administered to the cell culture. Contact can also be facilitated by incorporation of agent with carriers and delivery molecules and complexes, by injection, by infusion, etc.

After the agent has been administered in such a way that it can gain access to the cells, it can be determined whether the test agent modulates SEQ ID NOS 1-14 and 19-25 expression. Modulation can be of any type, quality, or quantity, e.g., increase, facilitate, enhance, up-regulate, stimulate, activate, amplify, augment, induce, decrease, down-regulate, diminish, lessen, reduce, etc. The modulatory quantity can also encompass any value, e.g., 1%, 5%, 10%, 50%, 75%, 1-fold, 2-fold, 5-fold, 10-fold, 100-fold, etc. To modulate SEQ ID NOS 1-14 and 19-25 expression means, e.g., that the test agent has an effect on its expression, e.g., to effect the amount of transcription, to effect RNA splicing, to effect translation of the RNA into polypeptide, to effect RNA or polypeptide stability, to effect polyadenylation or other processing of the RNA, to effect post-transcriptional or post-translational processing, etc.

A test agent can be of any molecular composition, e.g., chemical compounds, biomolecules, such as polypeptides, lipids, nucleic acids (e.g., antisense to a polynucleotide sequence selected from SEQ ID NOS 1-14 and 19-25), carbohydrates, antibodies, ribozymes, double-stranded RNA, etc. For example, if a gene to be modulated is a cell-surface molecule, a test agent can be an antibody that specifically recognizes it and leads to some effect on its expression. An antibody can cause the polypeptide to be internalized, leading to its down regulation on the surface of the cell. Such an effect does not have to be permanent, but can require the presence of the antibody to continue the down-regulatory effect. Antisense SEQ ID NOS 1-14 and 19-25 can also be used as test agents to modulate gene expression.

Markers

The polynucleotides of the present invention can be used with other markers, especially small intestine or colon markers, to identity, detect, stage, diagnosis, determine, prognosticate, treat, etc., tissue, diseases and conditions, etc, of the small intestine or colon. Markers can be polynucleotides, polypeptides, antibodies, ligands, specific binding partners, etc. The targets for such markers include, but are not limited genes and polypeptides that are selective for cell types present in the small intestine or colon. Specific targets include, e.g., genes of the cryptdin family (e.g., Ogawa et al., Am. J. Physiol. Gastrointest. Liver Physiol., 279:G492499, 2000; Darmul and Ouellette, Am. J. Physiol., 271:G68-7, 1996), guanylin (Steinbrecher et al., Biochem. Biophy. Res. Comm., 273:225-230, 2000), guanylate cyclase C, Mom1, neurotensin, intelectin (e.g., Komiya et al., Biochem. Biophy. Res. Comm., 251:759-762, 1998), nCL-4 (e.g., Lee et al., Biol. Chem., 379:175-183, 1998), MUC2, MUC3, CDx2 (e.g., Suh and Traber, Mol. Cell. Biol., 16:619-625, 1996), Min, endothelins, various functional enzymes and polypeptides, sich as calbindin, LPH, APN, AP, SGLT1, cryptdin, lysozyme, type II (secretory) phospholipase A2, intestinal defensin (e.g., RIP-3).

The polynucleotides of the present invention can be used with other markers, especially colon markers, to identity, detect, stage, diagnosis, determine, prognosticate, treat, etc., tissue, diseases and conditions, etc, of the colon. Markers can be polynucleotides, polypeptides, antibodies, ligands, specific binding partners, etc. The targets for such markers include, but are not limited genes and polypeptides that are selective for cell types present in the colon. Specific targets include, but are not limited to, carcinoembryonic antigen (CEA), alpha-fetoprotein (AFP), pancreatic oncofetal antigen (POA), lipid-bound sialic acid (LSA), myc, ras, TAG-72, p53, laminin-P1, urinary gonadotropin peptide (UGP), down regulated in adenoma (DRA, e.g., disclosed in U.S. Pat. No. 5,831,015), PP2R1B (Wang et al., Science, 282:284-2-87, 1998), adenomatous polyposis coli gene (APC), Asef (guanine nucleoide exchange factor associated with APC, e.g., Kawasaki et al., Science, 289:1194-1197, 2000) BAX (e.g., Rampino et al., Science, 275:967-969, 1998), Tcf genes (e.g., Korinek et al., Science, 275: 1784-1787, 1997), beta-catenin, Lef, Wnt, etc

Therapeutics

Selective polynucleotides, polypeptides, and specific-binding partners thereto, can be utilized in therapeutic applications, especially to treat diseases and conditions of small intestine or colon. Useful methods include, but are not limited to, immunotherapy (e.g., using specific-binding partners to polypeptides), vaccination (e.g., using a selective polypeptide or a naked DNA encoding such polypeptide), protein or polypeptide replacement therapy, gene therapy (e.g., germ-line correction, antisense), etc.

Various immunotherapeutic approaches can be used. For instance, unlabeled antibody that specifically recognizes a tissue-specific antigen can be used to stimulate the body to destroy or attack the cancer, to cause down-regulation, to produce complement-mediated lysis, to inhibit cell growth, etc., of target cells which display the antigen, e.g., analogously to how c-erbB-2 antibodies are used to treat breast cancer. In addition, antibody can be labeled or conjugated to enhance its deleterious effect, e.g., with radionuclides and other energy emitting entitities, toxins, such as ricin, exotoxin A (ETA), and diphtheria, cytotoxic or cytostatic agents, immunomodulators, chemotherapeutic agents, etc. See, e.g., U.S. Pat. No. 6,107,090.

An antibody or other specific-binding partner can be conjugated to a second molecule, such as a cytotoxic agent, and used for targeting the second molecule to a tissue-antigen positive cell (Vitetta, E. S. et al., 1993, Immunotoxin therapy, in DeVita, Jr., V. T. et al., eds, Cancer: Principles and Practice of Oncology, 4th ed., J. B. Lippincott Co., Philadelphia, 2624-2636). Examples of cytotoxic agents include, but are not limited to, antimetabolites, alkylating agents, anthracyclines, antibiotics, anti-mitotic agents, radioisotopes and chemotherapeutic agents. Further examples of cytotoxic agents include, but are not limited to ricin, doxorubicin, daunorubicin, taxol, ethidium bromide, mitomycin, etoposide, tenoposide, vincristine, vinblastine, colchicine, dihydroxy anthracin dione, actinomycin D, 1-dehydrotestosterone, diptheria toxin, Pseudomonas exotoxin (PE) A, PE40, abrin, elongation factor-2 and glucocorticoid. Techniques for conjugating therapeutic agents to antibodies are well.

In addition to immunotherapy, polynucleotides and polypeptides can be used as targets for non-immunotherapeutic applications, e.g., using compounds which interfere with function, expression (e.g., antisense as a therapeutic agent), assembly, etc. RNA interference can be used in vivtro and in vivo to silence SEQ ID NOS 1-14 and 19-25 when its expression contributes to a disease (but also for other purposes, e.g., to identify the gene's function to change a developmental pathway of a cell, etc.). See, e.g., Sharp and Zamore, Science, 287:2431-2433, 2001; Grishok et al., Science, 287:2494, 2001.

Delivery of therapeutic agents can be achieved according to any effective method, including, liposomes, viruses, plasmid vectors, bacterial delivery systems, orally, systemically, etc.

Antisense

Antisense polynucleotide (e.g., RNA) can also be prepared from a polynucleotide according to the present invention, preferably an anti-sense to a sequence of SEQ ID NOS 1-14 and 19-25. Antisense polynucleotide can be used in various ways, such as to regulate or modulate expression of the polypeptides they encode, e.g., inhibit their expression, for in situ hybridization, for therapeutic purposes, for making targeted mutations (in vivo, triplex, etc.) etc. For guidance on administering and designing anti-sense, see, e.g., U.S. Pat. Nos. 6,200,960, 6,200,807, 6,197,584, 6,190,869, 6,190,661, 6,187,587, 6,168,950, 6,153,595, 6,150,162, 6,133,246, 6,117,847, 6,096,722, 6,087,343, 6,040,296, 6,005,095,5,998,383, 5,994,230, 5,891,725, 5,885,970, and 5,840,708. An antisense polynucleotides can be operably linked to an expression control sequence. A total length of about 35 bp can be used in cell culture with cationic liposomes to facilitate cellular uptake, but for in vivo use, preferably shorter oligonucleotides are administered, e.g. 25 nucleotides.

Antisense polynucleotides can comprise modified, nonnaturally-occurring nucleotides and linkages between the nucleotides (e.g., modification of the phosphate-sugar backbone; methyl phosphonate, phosphorothioate, or phosphorodithioate linkages; and 2′-O-methyl ribose sugar units), e.g., to enhance in vivo or in vitro stability, to confer nuclease resistance, to modulate uptake, to modulate cellular distribution and compartmentalization, etc. Any effective nucleotide or modification can be used, including those already mentioned, as known in the art, etc., e.g., disclosed in U.S. Pat. Nos. 6,133,438; 6,127,533; 6,124,445; 6,121,437; 5,218,103 (e.g., nucleoside thiophosphoramidites); 4,973,679; Sproat et al., “2′-O-Methyloligoribonucleotides: synthesis and applications,” Oligonucleotides and Analogs A Practical Approach, Eckstein (ed.), IRL Press, Oxford, 1991, 49-86; Iribarren et al., “2′O-Alkyl Oligoribonucleotides as Antisense Probes,” Proc. Natl. Acad. Sci. USA, 1990, 87, 7747-7751; Cotton et al., “2′-O-methyl, 2′-O-ethyl oligoribonucleotides and phosphorothioate oligodeoxyribonucleotides as inhibitors of the in vitro U7 snRNP-dependent mRNA processing event,” Nucl. Acids Res., 1991, 19, 2629-2635.

Arrays

The present invention also relates to an ordered array of polynucleotide probes and specific-binding partners (e.g., antibodies) for detecting the expression of SEQ ID NOS 1-14 and 19-25 in a sample, comprising, one or more polynucleotide probes or specific binding partners associated with a solid support, wherein each probe is specific for SEQ ID NOS 1-14 and 19-25, and the probes comprise a nucleotide sequence of SEQ ID NOS 1-14 and 19-25 which is specific for said gene, a nucleotide sequence having sequence identity to SEQ ID NOS 1-14 and 19-25 which is specific for said gene or polynucleotide, or complements thereto, or a specific-binding partner which is specific for SEQ ID NOS 1-14 and 19-25.

The phrase “ordered array” indicates that the probes are arranged in an identifiable or position-addressable pattern, e.g., such as the arrays disclosed in U.S. Pat. Nos. 6,156,501, 6,077,673, 6,054,270, 5,723,320, 5,700,637, WO09919711, WO00023803. The probes are associated with the solid support in any effective way. For instance, the probes can be bound to the solid support, either by polymerizing the probes on the substrate, or by attaching a probe to the substrate. Association can be, covalent, electrostatic, noncovalent, hydrophobic, hydrophilic, noncovalent, coordination, adsorbed, absorbed, polar, etc. When fibers or hollow filaments are utilized for the array, the probes can fill the hollow orifice, be absorbed into the solid filament, be attached to the surface of the orifice, etc. Probes can be of any effective size, sequence identity, composition, etc., as already discussed.

Ordered arrays can further comprise polynucleotide probes or specific-binding partners which are specific for other genes, including genes specific for small intestine or colon, or disorders associated with small intestine or colon.

Transgenic Animals

The present invention also relates to transgenic animals comprising SEQ ID NOS 1-14 and 19-25 genes. Such genes, as discussed in more detail below, include, but are not limited to, functionally-disrupted genes, mutated genes, ectopically or selectively-expressed genes, inducible or regulatable genes, etc. These transgenic animals can be produced according to any suitable technique or method, including homologous recombination, mutagenesis (e.g., ENU, Rathkolb et al., Exp. Physiol., 85(6):635-644, 2000), and the tetracycline-regulated gene expression system (e.g., U.S. Pat. No. 6,242,667). The term “gene” as used herein includes any part of a gene, i.e., regulatory sequences, promoters, enhancers, exons, introns, coding sequences, etc. The SEQ ID NOS 1-14 and 19-25 nucleic acid present in the construct or transgene can be naturally-occurring wild-type, polymorphic, or mutated.

Along these lines, polynucleotides of the present invention can be used to create transgenic animals, e.g. a non-human animal, comprising at least one cell whose genome comprises a functional disruption of SEQ ID NOS 1-14 and 19-25. By the phrases “functional disruption” or “functionally disrupted,” it is meant that the gene does not express a biologically-active product. It can be substantially deficient in at least one functional activity coded for by the gene. Expression of a polypeptide can be substantially absent, i.e., essentially undetectable amounts are made. However, polypeptide can also be made, but which is deficient in activity, e.g., where only an amino-terminal portion of the gene product is produced.

The transgenic animal can comprise one or more cells. When substantially all its cells contain the engineered gene, it can be referred to as a transgenic animal “whose genome comprises” the engineered gene. This indicates that the endogenous gene loci of the animal has been modified and substantially all cells contain such modification.

Functional disruption of the gene can be accomplished in any effective way, including, e.g., introduction of a stop codon into any part of the coding sequence such that the resulting polypeptide is biologically inactive (e.g., because it lacks a catalytic domain, a ligand binding domain, etc.), introduction of a mutation into a promoter or other regulatory sequence that is effective to turn it off, or reduce transcription of the gene, insertion of an exogenous sequence into the gene which inactivates it (e.g., which disrupts the production of a biologically-active polypeptide or which disrupts the promoter or other transcriptional machinery), deletion of sequences from the SEQ ID NOS 1-14 and 19-25 gene, etc. Examples of transgenic animals having functionally disrupted genes are well known, e.g., as described in U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824. A transgenic animal which comprises the functional disruption can also be referred to as a “knock-out” animal, since the biological activity of its SEQ ID NOS 1-14 and 19-25 genes has been “knocked-out.” Knock-outs can be homozygous or heterozygous.

For creating functional disrupted genes, and other gene mutations, homologous recombination technology is of special interest since it allows specific regions of the genome to be targeted. Using homologous recombination methods, genes can be specifically-inactivated, specific mutations can be introduced, and exogenous sequences can be introduced at specific sites. These methods are well known in the art, e.g., as described in the patents above. See, also, Robertson, Biol. Reproduc., 44(2):238-245, 1991. Generally, the genetic engineering is performed in an embryonic stem (ES) cell, or other pluripotent cell line (e.g., adult stem cells, EG cells), and that genetically-modified cell (or nucleus) is used to create a whole organism. Nuclear transfer can be used in combination with homologous recombination technologies.

For example, the SEQ ID NOS 1-14 and 19-25 locus can be disrupted in mouse ES cells using a positive-negative selection method (e.g., Mansour et al., Nature, 336:348-352, 1988). In this method, a targeting vector can be constructed which comprises a part of the gene to be targeted. A selectable marker, such as neomycin resistance genes, can be inserted into a SEQ ID NOS 1-14 and 19-25 exon present in the targeting vector, disrupting it. When the vector recombines with the ES cell genome, it disrupts the function of the gene. The presence in the cell of the vector can be determined by expression of neomycin resistance. See, e.g., U.S. Pat. No. 6,239,326. Cells having at least one functionally disrupted gene can be used to make chimeric and germline animals, e.g., animals having somatic and/or germ cells comprising the engineered gene. Homozygous knock-out animals can be obtained from breeding heterozygous knock-out animals. See, e.g., U.S. Pat. No. 6,225,525. A transgenic animal, or animal cell, lacking one or more functional SEQ ID NOS 1-14 and 19-25 genes can be useful in a variety of applications, including, as an animal model for small intestine or colon diseases, for drug screening assays (e.g., by making a cell deficient in SEQ ID NOS 1-14 and 19-25, the contribution of other sequences can be specifically examined), as a source of tissues deficient in SEQ ID NOS 1-14 and 19-25 activity, and any of the utilities mentioned in any issued U.S. Patent on transgenic animals, including, U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824.

The present invention also relates to non-human, transgenic animal whose genome comprises recombinant SEQ ID NOS 1-14 and 19-25 nucleic acid operatively linked to an expression control sequence effective to express said coding sequence, e.g., in small instestine. such a transgenic animal can also be referred to as a “knock-in” animal since an exogenous gene has been introduced, stably, into its genome.

A recombinant SEQ ID NOS 1-14 and 19-25 nucleic acid refers to a gene which has been introduced into a target host cell and optionally modified, such as cells derived from animals, plants, bacteria, yeast, etc. A recombinant SEQ ID NOS 1-14 and 19-25 includes completely synthetic nucleic acid sequences, semi-synthetic nucleic acid sequences, sequences derived from natural sources, and chimeras thereof. “Operable linkage” has the meaning used through the specification, i.e., placed in a functional relationship with another nucleic acid. When a gene is operably linked to an expression control sequence, as explained above, it indicates that the gene (e.g., coding sequence) is joined to the expression control sequence (e.g., promoter) in such a way that facilitates transcription and translation of the coding sequence. As described above, the phrase “genome” indicates that the genome of the cell has been modified. In this case, the recombinant SEQ ID NOS 1-14 and 19-25 has been stably integrated into the genome of the animal. The SEQ ID NOS 1-14 and 19-25 nucleic acid in operable linkage with the expression control sequence can also be referred to as a construct or transgene.

Any expression control sequence can be used depending on the purpose. For instance, if selective expression is desired, then expression control sequences which limit its expression can be selected. These include, e.g., tissue or cell-specific promoters, introns, enhancers, etc. For various methods of cell and tissue-specific expression, see, e.g., U.S. Pat. Nos. 6,215,040, 6,210,736, and 6,153,427. These also include the endogenous promoter, i.e., the coding sequence can be operably linked to its own promoter. Inducible and regulatable promoters can also be utilized.

The present invention also relates to a transgenic animal which contains a functionally disrupted and a transgene stably integrated into the animals genome. Such an animal can be constructed using combinations any of the above- and below-mentioned methods. Such animals have any of the aforementioned uses, including permitting the knock-out of the normal gene and its replacement with a mutated gene. Such a transgene can be integrated at the endogenous gene locus so that the functional disruption and “knock-in” are carried out in the same step.

In addition to the methods mentioned above, transgenic animals can be prepared according to known methods, including, e.g., by pronuclear injection of recombinant genes into pronuclei of 1-cell embryos, incorporating an artificial yeast chromosome into embryonic stem cells, gene targeting methods, embryonic stem cell methodology, cloning methods, nuclear transfer methods. See, also, e.g., U.S. Pat. Nos. 4,736,866; 4,873,191; 4,873,316; 5,082,779; 5,304,489; 5,174,986; 5,175,384; 5,175,385; 5,221,778; Gordon et al., Proc. Natl. Acad. Sci., 77:7380-7384, 1980; Palmiter et al., Cell, 41:343-345, 1985; Palmiter et al., Ann. Rev. Genet, 20:465499, 1986; Askew et al., Mol. Cell. Bio., 13:4115-4124, 1993; Games et al. Nature, 373:523-527, 1995; Valancius and Smithies, Mol. Cell. Bio., 11:1402-1408, 1991; Stacey et al., Mol. Cell. Bio., 14:1009-1016, 1994; Hasty et al., Nature, 350:243-246, 1995; Rubinstein et al., Nucl. Acid Res., 21:2613-2617,1993; Cibelli et al., Science, 280:1256-1258, 1998. For guidance on recombinase excision systems, see, e.g., U.S. Pat. Nos. 5,626,159, 5,527,695, and 5,434,066. See also, Orban, P. C., et al., “Tissue- and Site-Specific DNA Recombination in Transgenic Mice,” Proc. Natl. Acad. Sci. USA, 89:6861-6865 (1992); O'Gorman, S., et al., “Recombinase-Mediated Gene Activation and Site-Specific Integration in Mammalian Cells,” Science, 251:1351-1355 (1991); Sauer, B., et al., “Cre-stimulated recombination at loxP-Containing DNA sequences placed into the mammalian genome,” Polynucleotides Research, 17(1):147-161 (1989); Gagneten, S. et al. (1997) Nucl. Acids Res. 25:3326-3331; Xiao and Weaver (1997) Nucl. Acids Res. 25:2985-2991; Agah, R. et al. (1997) J. Clin. Invest. 100:169-179; Barlow, C. et al. (1997) Nucl. Acids Res. 25:2543-2545; Araki, K. et al. (1997) Nucl. Acids Res. 25:868-872; Mortensen, R. N. et al. (1992) Mol. Cell. Biol. 12:2391-2395 (G418 escalation method); Lalkhlani, P. P. et al. (1997) Proc. Natl. Acad. Sci. USA 94:9950-9955 (“hit and run”); Westphal and Leder (1997) Curr. Biol. 7:530-533 (transposon-generated “knock-out” and “knock-in”); Templeton, N. S. et al. (1997) Gene Ther. 4:700-709 (methods for efficient gene targeting, allowing for a high frequency of homologous recombination events, e.g., without selectable markers); PCT International Publication WO 93/22443 (functionally-disrupted).

A polynucleotide according to the present invention can be introduced into any non-human animal, including a non-human mammal, mouse (Hogan et al., Manipulating the Mouse Embryo: A Laboratory Manual, Cold Spring Harbor Laboratory, Cold Spring Harbor, N.Y., 1986), pig (Hammer et al., Nature, 315:343-345, 1985), sheep (Hammer et al., Nature, 315:343-345, 1985), cattle, rat, or primate. See also, e.g., Church, 1987, Trends in Biotech. 5:13-19; Clark et al., Trends in Biotech. 5:20-24, 1987); and DePamphilis et al., BioTechniques, 6:662-680, 1988. Transgenic animals can be produced by the methods described in U.S. Pat. No. 5,994,618, and utilized for any of the utilities described therein.

Database

The present invention also relates to electronic forms of polynucleotides, polypeptides, etc., of the present invention, including computer-readable medium (e.g., magnetic, optical, etc., stored in any suitable format, such as flat files or hierarchical files) which comprise such sequences, or fragments thereof, e-commerce-related means, etc. Along these lines, the present invention relates to methods of retrieving gene sequences from a computer-readable medium, comprising, one or more of the following steps in any effective order, e.g., selecting a cell or gene expression profile, e.g., a profile that specifies that said gene is differentially expressed in small intestine or colon, and retrieving said differentially expressed gene sequences, where the gene sequences consist of the genes represented by SEQ ID NOS 1-14 and 19-25.

A “gene expression profile” means the list of tissues, cells, etc., in which a defined gene is expressed (i.e, transcribed and/or translated). A “cell expression profile” means the genes which are expressed in the particular cell type. The profile can be a list of the tissues in which the gene is expressed, but can include additional information as well, including level of expression (e.g., a quantity as compared or normalized to a control gene), and information on temporal (e.g., at what point in the cell-cycle or developmental program) and spatial expression. By the phrase “selecting a gene or cell expression profile,” it is meant that a user decides what type of gene or cell expression pattern he is interested in retrieving, e.g., he may require that the gene is differentially expressed in a tissue, or he may require that the gene is not expressed in blood, but must be expressed in small intestine. Any pattern of expression preferences may be selected. The selecting can be performed by any effective method. In general, “selecting” refers to the process in which a user forms a query that is used to search a database of gene expression profiles. The step of retrieving involves searching for results in a database that correspond to the query set forth in the selecting step. Any suitable algorithm can be utilized to perform the search query, including algorithms that look for matches, or that perform optimization between query and data. The database is information that has been stored in an appropriate storage medium, having a suitable computer-readable format. Once results are retrieved, they can be displayed in any suitable format, such as HTML.

For instance, the user may be interested in identifying genes that are differentially expressed in a small intestine or colon. He may not care whether small amounts of expression occur in other tissues, as long as such genes are not expressed in peripheral blood lymphocytes. A query is formed by the user to retrieve the set of genes from the database having the desired gene or cell expression profile. Once the query is inputted into the system, a search algorithm is used to interrogate the database, and retrieve results.

Advertising, Licensing, etc., Methods

The present invention also relates to methods of advertising, licensing, selling, purchasing, brokering, etc., genes, polynucleotides, specific-binding partners, antibodies, etc., of the present invention. Methods can comprises, e.g., displaying a SEQ ID NOS 1-14 and 19-25 gene, SEQ ID NOS 1-14 and 19-25 polypeptide, or antibody specific for SEQ ID NOS 1-14 and 19-25 in a printed or computer-readable medium (e.g., on the Web or Internet), accepting an offer to purchase said gene, polypeptide, or antibody.

Other

A polynucleotide, probe, polypeptide, antibody, specific-binding partner, etc., according to the present invention can be isolated. The term “isolated” means that the material is in a form in which it is not found in its original environment or in nature, e.g., more concentrated, more purified, separated from component, etc. An isolated polynucleotide includes, e.g., a polynucleotide having the sequenced separated from the chromosomal DNA found in a living animal, e.g., as the complete gene, a transcript, or a cDNA. This polynucleotide can be part of a vector or inserted into a chromosome (by specific gene-targeting or by random integration at a position other than its normal position) and still be isolated in that it is not in a form that is found in its natural environment. A polynucleotide, polypeptide, etc., of the present invention can also be substantially purified. By substantially purified, it is meant that polynucleotide or polypeptide is separated and is essentially free from other polynucleotides or polypeptides, i.e., the polynucleotide or polypeptide is the primary and active constituent. A polynucleotide can also be a recombinant molecule. By “recombinant,” it is meant that the polynucleotide is an arrangement or form which does not occur in nature. For instance, a recombinant molecule comprising a promoter sequence would not encompass the naturally-occurring gene, but would include the promoter operably linked to a coding sequence not associated with it in nature, e.g., a reporter gene, or a truncation of the normal coding sequence.

The term “marker” is used herein to indicate a means for detecting or labeling a target. A marker can be a polynucleotide (usually referred to as a “probe”), polypeptide (e.g., an antibody conjugated to a detectable label), PNA, or any effective material.

The topic headings set forth above are meant as guidance where certain information can be found in the application, but are not intended to be the only source in the application where information on such topic can be found. Reference materials

For other aspects of the polynucleotides, reference is made to standard textbooks of molecular biology. See, e.g., Hames et al., Polynucleotide Hybridization, IL Press, 1985; Davis et al., Basic Methods in Molecular Biology, Elsevir Sciences Publishing, Inc., New York, 1986; Sambrook et al., Molecular Cloning, CSH Press, 1989; Howe, Gene Cloning and Manipulation, Cambridge University Press, 1995; Ausubel et al., Current Protocols in Molecular Biology, John Wiley & Sons, Inc., 1994-1998.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever. The entire disclosure of all applications, patents and publications, cited above and in the figures are hereby incorporated by reference in their entirety.

TABLE 1

| | Type of | Clone seq | |
|---|---|---|---|
| Clone name | Polymorphism | (Pos/nt) | Genomic Seq. (Accn#, nt) |
| SI58 | Substitution | 303, C | NT_010441, T |
| | Substitution | 1649, C | AC003034, T |
| SI61 | Substitution | 1889,T | AL021939, C |
| SI085 | Substitution | 550, C | AL021939, T |
| | Insertion | 2385, A | AL021939, * |

1. An isolated polynucleotide comprising,
a polynucleotide sequence set forth in SEQ ID NOS 1-14, 19-24, or 25, or a complement thereto.

2. An isolated polynucleotide of claim 1 which codes without interruption for an amino acid sequence set forth in SEQ ID NOS 1-14, 19-24, or 25, or a complement thereto.

3. An isolated polynucleotide comprising,
polynucleotide sequence having 95% or more sequence identity to the polynucleotide sequence set forth in SEQ ID NOS 1-14, 19-24, or 25 and which codes without interruption for SEQ ID NOS 1-14, 19-24, or 25, or a complement thereto.

4. An isolated polynucleotide of claim 3 which is selective for small intestine or colon.

5. An isolated polynucleotide of claim 1 consisting of:
SEQ ID NOS 1-14, 19-24, or 25, or a complement thereof.

6. An isolated polynucleotide consisting of:
a polynucleotide fragment of SEQ ID NO 1-14, 19-24, or 25 which is specific for SEQ ID NO 1-14, 19-24, or 25, or a complement thereof.

7. An isolated polynucleotide of claim 6, wherein said fragment is effective in a polymerase chain reaction.

8. An isolated polypeptide
coded for by a polynucleotide of claim 1.

9. An isolated polypeptide, selected from the amino acid sequence of SEQ ID NOS 1-14, 19-24, or 25 of claim 8 which is specific-for a polypeptide selected from SEQ ID NOS 1-14, 19-24, or 25.

10. An isolated polypeptide comprising an amino acid sequence having 95% or more sequence identity to the polypeptide of claim 8.

11. A method of diagnosing a small intestine or colon disease associated with abnormal SEQ ID NO 1-14, 19-24, or 25, comprising:
assessing the expression of SEQ ID NO 1-14, 19-24, or 25 of claim 1 in a tissue sample comprising small intestine or colon, or cells derived from small intestine or colon.

12. A method of claim 1, wherein assessing is:
measuring expression levels of said gene, determining the genomic structure of said gene, determining the mRNA structure of transcripts from said gene, or measuring the expression levels of polypeptide coded for by said gene.

13. A method of claim 12, further comprising:
comparing said expression to the expression of said gene of a known normal tissue.

14. A method of claim 1, wherein said assessing detecting is performed by:
Northern blot analysis, polymerase chain reaction (PCR), reverse transcriptase PCR, RACE PCR, or in situ hybridization, and
using a polynucleotide probe having a sequence selected from SEQ ID NO 1-14, 19-24, or 25, a polynucleotide having 95% sequence identity or more to a sequence set forth in SEQ ID NO 1-14, 19-24, or 25, effective specific fragments thereof, or complements thereto.

15. A method of assessing a therapeutic or preventative intervention in a subject having a small intestine or colon disease, comprising,
determining the expression levels of SEQ ID NO 1-14, 19-24, or 25 of claim 1 in a tissue sample comprising small intestine or colon cells, or cells derived from small intestine or colon.

16 (cancelled).

17. A method of detecting a nucleic acid coding for a polynucleotide of claim 1, comprising,
contacting a sample comprising nucleic acid with a polynucleotide probe specific for a polynucleotide of claim 1 under conditions effective for said probe to hybridize specifically with said gene, and
detecting hybridization between said probe and said nucleic acid.

18. An antibody which is specific-for a polypeptide of claim 8.

19 (cancelled).

20 (cancelled).

21 (cancelled).
